SEQUENCE LISTING (9 sequences)

SEQ ID NO: 1 - DNA, 2576 bp, Homo sapiens
gaattccgggcgacgcgcgggaacaacgcgagtcggcgcgcgggacgaagaataatcatgggccagactgggaagaaatctgagaagggaccagtttgttggcggaagcgtgtaaaatcagagtacatgcgactgagacagctcaagaggttcagacgagctgatgaagtaaagagtatgtttagttccaatcgtcagaaaattttggaaagaacggaaatcttaaaccaagaatggaaacagcgaaggatacagcctgtgcacatcctgacttctgtgagctcattgcgcgggactagggagtgttcggtgaccagtgacttggattttccaacacaagtcatcccattaaagactctgaatgcagttgcttcagtacccataatgtattcttggtctcccctacagcagaattttatggtggaagatgaaactgttttacataacattccttatatgggagatgaagttttagatcaggatggtactttcattgaagaactaataaaaaattatgatgggaaagtacacggggatagagaatgtgggtttataaatgatgaaatttttgtggagttggtgaatgcccttggtcaatataatgatgatgacgatgatgatgatggagacgatcctgaagaaagagaagaaaagcagaaagatctggaggatcaccgagatgataaagaaagccgcccacctcggaaatttccttctgataaaattttggaggccatttcctcaatgtttccagataagggcacagcagaagaactaaaggaaaaatataaagaactcaccgaacagcagctcccaggcgcacttcctcctgaatgtacccccaacatagatggaccaaatgctaaatctgttcagagagagcaaagcttacactcctttcatacgcttttctgtaggcgatgttttaaatatgactgcttcctacatccttttcatgcaacacccaacacttataagcggaagaacacagaaacagctctagacaacaaaccttgtggaccacagtgttaccagcatttggagggagcaaaggagtttgctgctgctctcaccgctgagcggataaagaccccaccaaaacgtccaggaggccgcagaagaggacggcttcccaataacagtagcaggcccagcacccccaccattaatgtgctggaatcaaaggatacagacagtgatagggaagcagggactgaaacggggggagagaacaatgataaagaagaagaagagaagaaagatgaaacttcgagctcctctgaagcaaattctcggtgtcaaacaccaataaagatgaagccaaatattgaacctcctgagaatgtggagtggagtggtgctgaagcctcaatgtttagagtcctcattggcacttactatgacaatttctgtgccattgctaggttaattgggaccaaaacatgtagacaggtgtatgagtttagagtcaaagaatctagcatcatagctccagctcccgctgaggatgtggatactcctccaaggaaaaagaagaggaaacaccggttgtgggctgcacactgcagaaagatacagctgaaaaaggacggctcctctaaccatgtttacaactatcaaccctgtgatcatccacggcagccttgtgacagttcgtgcccttgtgtgatagcacaaaatttttgtgaaaagttttgtcaatgtagttcagagtgtcaaaaccgctttccgggatgccgctgcaaagcacagtgcaacaccaagcagtgcccgtgctacctggctgtccgagagtgtgaccctgacctctgtcttacttgtggagccgctgaccattgggacagtaaaaatgtgtcctgcaagaactgcagtattcagcggggctccaaaaagcatctattgctggcaccatctgacgtggcaggctgggggatttttatcaaagatcctgtgcagaaaaatgaattcatctcagaatactgtggagagattatttctcaagatgaagctgacagaagagggaaagtgtatgataaatacatgtgcagctttctgttcaacttgaacaatgattttgtggtggatgcaacccgcaagggtaacaaaattcgttttgcaaatcattcggtaaatccaaactgctatgcaaaagttatgatggttaacggtgatcacaggataggtatttttgccaagagagccatccagactggcgaagagctgtttgttgattacagatacagccaggctgatgccctgaagtatgtcggcatcgaaagagaaatggaaatcccttgacatctgctacctcctccccctcctctgaaacagctgccttagcttcaggaacctcgagtactgtgggcaatttagaaaaagaacatgcagtttgaaattctgaatttgcaaagtactgtaagaataatttatagtaatgagtttaaaaatcaactttttattgccttctcaccagctgcaaagtgttttgtaccagtgaatttttgcaataatgcagtatggtacatttttcaactttgaataaagaatacttgaacttgaaaaaaaaaaaaaaaaaa

SEQ ID NO: 2 - protein, 746 aa, Homo sapiens
MGQTGKKSEKGPVCWRKRVKSEYMRLRQLKRFRRADEVKSMFSSNRQKILERTEILNQEWKQRRIQPVHILTSVSSLRGTRECSVTSDLDFPTQVIPLKTLNAVASVPIMYSWSPLQQNFMVEDETVLHNIPYMGDEVLDQDGTFIEELIKNYDGKVHGDRECGFINDEIFVELVNALGQYNDDDDDDDGDDPEEREEKQKDLEDHRDDKESRPPRKFPSDKILEAISSMFPDKGTAEELKEKYKELTEQQLPGALPPECTPNIDGPNAKSVQREQSLHSFHTLFCRRCFKYDCFLHPFHATPNTYKRKNTETALDNKPCGPQCYQHLEGAKEFAAALTAERIKTPPKRPGGRRRGRLPNNSSRPSTPTINVLESKDTDSDREAGTETGGENNDKEEEEKKDETSSSSEANSRCQTPIKMKPNIEPPENVEWSGAEASMFRVLIGTYYDNFCAIARLIGTKTCRQVYEFRVKESSIIAPAPAEDVDTPPRKKKRKHRLWAAHCRKIQLKKDGSSNHVYNYQPCDHPRQPCDSSCPCVIAQNFCEKFCQCSSECQNRFPGCRCKAQCNTKQCPCYLAVRECDPDLCLTCGAADHWDSKNVSCKNCSIQRGSKKHLLLAPSDVAGWGIFIKDPVQKNEFISEYCGEIISQDEADRRGKVYDKYMCSFLFNLNNDFVVDATRKGNKIRFANHSVNPNCYAKVMMVNGDHRIGIFAKRAIQTGEELFVDYRYSQADALKYVGIEREMEIP

SEQ ID NO: 3 - protein, 6 aa, Artificial Sequence
  Homo sapiens
TKQTAR

SEQ ID NO: 4 - protein, 9 aa, Artificial Sequence
  Homo sapiens
KSTGGKAPR

SEQ ID NO: 5 - protein, 9 aa, Artificial Sequence
  Homo sapien
KQLATKAAR

SEQ ID NO: 6 - protein, 14 aa, Artificial Sequence
  Homo sapiens
KSAPATGGVKKPHR

SEQ ID NO: 7 - protein, 9 aa, Artificial Sequence
  Homo sapiens
YRPGTVALR

SEQ ID NO: 8 - protein, 10 aa, Artificial Sequence
  Homo sapiens
YQKSTELLIR

SEQ ID NO: 9 - protein, 11 aa, Artificial Sequence
  Homo sapiens
EIAQDFKTDLR

(19) United States
(12) Patent Application Publication
(10) Pub. No.: US 2016/0091485 A1
(43) Pub. Date: Mar. 31, 2016

(54) MARKERS FOR EZH2 INHIBITORS

(71) Applicants: Ho Man CHAN, (US); Veronica GIBAJA, Cambridge, MA (US); Kristy HAAS, Cambridge, MA (US); Jacob JAFFE, Boston, MA (US); Mariela JASKELIOFF, Cambridge, MA (US); Frank STEGMEIER, Cambridge, MA (US); Wei QI, Shanghai (CN); THE BROAD INSTITUTE, INC., Cambridge, MA (US)

(72) Inventors: Ho Man CHAN, Arlington, CA (US); Veronica GIBAJA, Dorchester, MA (US); Kristy HAAS, Brighton, MA (US); Jacob JAFFE, Cambridge, MA (US); Mariela JASKELIOFF, Roslindale, MA (US); Frank Peter STEGMEIER, Cambridge, MA (US); Wei QI, Shanghai, CN (US)

(73) Assignees: THE BROAD INSTITUTE, INC., Cambridge, MA (US); NOVARTIS AG, Basel (CH)

(21) Appl. No.: 14/785,594

(22) PCT Filed: Apr. 11, 2014
(86) PCT No.: PCT/US2014/033786

§ 371 Date: Oct. 19, 2015

(30) Foreign Application Priority Data
Apr. 25, 2013 (CN) PCT/CN2013/074699

(51) Int. Cl. G01N 33/50

(52) U.S. Cl. CPC G01N 33/5011; G01N 2560/00

(57) ABSTRACT

The invention provides methods of detecting an EZH2 mutation and associated epigenetic markers in a cancer cell, methods cancer diagnosis and methods of screening for EZH2 inhibitors.

### FIELD OF THE INVENTION

The present invention relates to the field of pharmacogenomics, and the use of biomarkers useful in determining patient sensitivity prior to treatment, following patient response after treatment, cancer sensitivity and screening of compounds.

### BACKGROUND

Enhancer of Zeste is a gene found in Drosophila development which represses gene expression and plays a role in body segmentation of the animal. The mammalian homologue of this gene is known as EZH2. EZH2 is part of a heterotrimeric complex of proteins which has histone methyltransferase activity and acts on histone H3 at lysine 27 (H3K27) (Cao et al., Science 2000; 298(5595):1039-1043, Morey et al., Trends Biochem. Sci. 2010; 35(6):323-332). As to the role of EZH2 in normal development, the EZH2 knockout mice are embryonic lethal (O'Carroll et al., Mol. Cell Bio. 2001; 21(13):4330-4336).

The wild type EZH2 is an efficient mono-methyltranferase (me1)) in vitro, however it is only weakly processive for adding a second (me2) and especially weak for adding a third (me3) methyl group. In cancers, point mutations in EZH2 have been described as gain of function mutations that increase the catalytic efficiency of di-methyl to tri-methyl reactions (Yap et al., Blood 2011; 117:2451-2459, Sneeringer et al., Proc. Natl, Acad, Sci. USA 2010 107:20980). However, these EZH2 gain of function mutations render the enzyme inactive as a mono-methyltransferase. The EZH2 gain-of-function mutations are found at tyrosine 641, with changes to phenylalanine (F), serine (S), asparagine (N), histidine (H), cysteine (C) (Morin et al., Nat. Genet. 2010; 42:181-185, Morin et al., Nature 2011; 476:298-303). In addition, an alanine to glycine change has been noted at amino acid 677 (McCabe et al., Proc. Natl. Acad. Sci. USA 2012; 109:2989) and a alanine to valine change at 687 (Majer et al., Febs Let. 2012 586(19):3448-3451).

EZH2 mutations are found in diffuse large B cell lymphoma (DLBCL), (the Morin references, supra; Lohr et al., Proc. Natl. Acad. Sci. USA 2012; 109:3979-3884; Bodor et al., Leukemia 2011; 25(4):726-729). Mutations in EZH2 are always heterozygous when found in primary tumor cells from patients and it has been hypothesized that the wild type EZH2 and mutant EZH2 act together to maintain a high level of tri-methylation at histone 3, lysine 27 (H3K27me3) (Yap et al., Blood 2011; 117(8):2451-2459, Ryan et al., PLoS One 2011, 6(12) published online Dec. 14, 2011).

As reported in the literature cited above, EZH2 gain of function mutations correlate with an increase in H3K27me3. However, in this work, assaying for specific histone modifications and clustering those modifications provide a more accurate readout of patient insensitivity to EZH2 inhibitors than assaying for EZH2 mutations.

In this work, the methylation status is used as a biomarker or indicator. Finding biomarkers which indicate which patient should receive a therapeutic is useful, especially with regard to cancer. This allows for more timely and aggressive treatment as opposed to a trial and error approach. In addition, the discovery of biomarkers which indicate the patient is insensitive to the therapy is also useful. These biomarkers can be used to segregate the patient population. If biomarkers indicate that the patient is insensitive to the proposed treatment, then another therapeutic can be administered. As such, changes in the histone methylation pattern provide a method of determining when a patient is sensitive or insensitive to treatment with an EZH2 inhibitor. This approach ensures that the correct patients receive the appropriate treatment.

In the diagnosis of cancer, methylation biomarkers associated with EZH2 mutations will aid in formulating the treatment regimen. This analysis is done in order to determine the particular sensitivity of cancer cells containing an EZH2 mutation and the activity of the therapy.

### SUMMARY OF THE INVENTION

The disclosure is directed to diagnosis of cancer by analysis of histone modification. Epigenetic dysregulation of histone marks occurs commonly in cancer. The mass spectrometry (MS) approach described herein provides an unbiased method to systematically functionally annotate chromatin state, which we called molecular chromatin signature (MCS). This method circumvents many limitations of antibody-based methods to profile global histone modifications, which are highly dependent on the availability and specificity of the antibody reagents which are often cross-reactive (Peach et al., Mol Cell Prot. 2012 11:128-137). In addition, the method as described herein provides a more accurate readout of resistance to EZH2 inhibitors than genotyping. The method analyzes a MCS in a cancer sample taken from a patient and compared to a MCS in a non-mutant or wild-type control. The MCS pattern can be indicative of a favorable response or an unfavorable one. The invention is an example of “personalized medicine” wherein patients are treated based on a functional genomic signature that is specific to that individual.

The predictive value of MCS profiling is especially useful in segregating patients that would be insensitive to EZH2 inhibitor treatment. This is useful in determining that patients receive the correct course of treatment.

### DESCRIPTION OF THE DRAWINGS

FIG. 1 is a heat map of histone mass spectrometry profiling and clustered by their epigenetic differences.

FIG. 2A shows a heat map of histone profiling associated with EZH2 gain of function mutations, cluster A.

FIG. 2B shows a heat map of histone profiling associated with EZH2 loss of function mutations, cluster F.

FIG. 3 is a table that provides the clustering of cell lines according to their MCS and associated EZH2 mutations.

FIG. 4 is a table that shows cell type and sensitivity to an EZH2 inhibitor as grouped by EZH2 mutant status and by histone profile cluster.

FIG. 5 shows that cells with a particular MCS (Cluster A) are the most sensitive to EZH2 inhibitors.

FIG. 6 shows that cells carrying EZH2 gain of function mutation, with a different MCS (not in cluster A) are insensitive to EZH2 inhibitors.

### DESCRIPTION OF THE INVENTION

The disclosure is directed to a method of detecting sensitivity of a cancer cell to an EZH2 inhibitor, the method comprising; a) obtaining a cancer sample from a patient; b) isolating histones from the cancer sample; c) analyzing the histones by mass spectrometry to obtain a molecular chromatin signal (MCS); and d) comparing the MCS of the cancer sample to a wild-type MCS in a non-cancerous or normal patient sample.

The method wherein the cancer cell contains a mutation in EZH2.

The method wherein the mutation in EZH2 is a change from alanine (A) at amino acid position 677 to a glycine (G); the mutation in EZH2 is a change from tyrosine (Y) at amino acid position 641 to the group consisting of serine (S), cysteine (C), phenylalanine (F), histidine (H) and asparagine (N), or the mutation in EZH2 is a change from alanine (A) at amino acid position 687 to a valine (V).

The method wherein the MCS is tri-methylation at histone H3, lysine 27 (H3K27me3).

The method wherein the H3K27me3 level is low or decreased, indicating insensitivity of the cancer cell to an EZH2 inhibitor.

The method, wherein the cancer cell is selected from the group consisting of: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

The method further comprising: e) contacting cells of the cancer sample with an EZH2 inhibitor of the pyridinonyl substituted indolines class of molecules to determine sensitivity.

The method wherein the EZH2 inhibitor is 6-cyano-N-((4,6-dimethyl-2-oxo-1,2-dihydropyridin-3-yl)methyl)-1-(pentan-3-yl)-1H-indole-4-carboxamide (EI1).

A method of screening for an EZH2 inhibitor candidate, the method comprising: a) contacting a cell containing an EZH2 gain of function mutation with an EZH2 inhibitor candidate; b) isolating histones from the contacted cell; c) analyzing the histones by mass spectrometry to obtain a molecular chromatin signal (MCS); and d) comparing the reduction in H3K27me3 level in the EZH2 mutant cell contacted with the EZH2 inhibitor candidate with the H3K27me3 level in a normal or control cell and/or untreated cells containing the EZH2 mutation.

The method wherein the mutation in EZH2 is a change from alanine (A) at amino acid position 677 to a glycine (G); the mutation in EZH2 is a change from tyrosine (Y) at amino acid position 641 to the group consisting of serine (S), cysteine (C), phenylalanine (F), histidine (H) and asparagine (N), or the mutation in EZH2 is a change from alanine (A) at amino acid position 687 to a valine (V).

The method, wherein the cell containing an EZH2 mutation is selected from the group consisting of: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

The method further comprising: e) contacting the EZH2 mutant cell with a molecule from the pyridinonyl substituted indolines class of molecules and comparing sensitivity to the EZH2 inhibitor candidate.

The method wherein the molecule from the pyridinonyl substituted indolines class of molecules is 6-cyano-N-((4,6-dimethyl-2-oxo-1,2-dihydropyridin-3-yl)methyl)-1-(pentan-3-yl)-1H-indole-4-carboxamide (EI1).

Composition comprising H3K27me3 for use in diagnosis of cancer in a selected cancer patient population, wherein the cancer patient population is selected on the basis of containing an EZH2 mutation in a cancer cell sample obtained from said patients compared to a normal control cell sample.

The composition wherein the cancer sample is selected from the group consisting of is selected from the group consisting of: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

A kit for predicting the insensitivity of a cancer patient for treatment with a EZH2 inhibitor comprising: i) means for detecting decreased levels of H3K27me3 by mass spectrometry; and ii) instructions how to use said kit.

### DEFINITIONS

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such are known in the art.

“EZH2” refers to the Enhancer of Zeste homolog 2 gene, a histone-lysine N-methyltransferase. Unless specifically stated otherwise EZH2 as used herein, refers to human EZH2, accession number X95653 (DNA (SEQ ID NO. 1)) and (protein (SEQ ID NO.2)).

The terms “marker” or “biomarker” are used interchangeably herein. A biomarker is a nucleic acid or polypeptide and the presence or absence of a mutation or differential expression is used to determine a specific cancer type. A biomarker can also include a post translational modification, for example phosphorylation or methylation of a particular protein at a particular amino acid.

“Molecular chromatin signature” (MCS) is a biomarker of histone methylation in a cancer cell when compared to the MCS in normal (non-cancerous) tissue or control tissue. For example, decreased tri-methylation at histone H3, lysine 27, (H3K27me3) is an MCS for EZH2 inhibitor resistant cells.

“Methylation” is the modification of amino acids on a histone protein by the addition of a methyl group. The amino acid can have no methylation (me0), have a single methyl group added (me1), two methyl groups added (me2) or three methyl groups (me3). For example, the nomenclature “H3K27me3” indicates that 3 methyl groups were added to histone H3 at the lysine at position 27. The “methylation status” or “methylation profile” refers to the histone, the amino acid and 0-3 methyl group modifications (me0-me3).

A cell is “sensitive” or displays “sensitivity” for inhibition with an EZH2 inhibitor when the methyltransferase activity of EZH2 is reduced compared to cells treated without an EZH2 inhibitor, or when cell proliferation is reduced. A cell is “insensitive” to an EZH2 inhibitor when no change in methlytransferase activity is observed or when cell proliferation is not affected.

A “mutant,” or “mutation” is any change in DNA or protein sequence that deviates from wild type EZH2. This includes single base DNA changes, single amino acid changes, multiple base changes in DNA and multiple amino acid changes. This also includes insertions, deletions and truncations of the EZH2 gene and its corresponding protein. For example, a mutation can be a tyrosine to serine change at amino acid position 641 (Y641S).

A “control cell,” “normal cell” or “wild-type” refers to non-cancerous cell.

A “control tissue,” “normal tissue” or “wild-type” refers to non-cancerous tissue.

The terms “nucleic acid” and “polynucleotide” are used interchangeably and refer to a polymeric form of nucleotides of any length, either deoxyribonucleotides or ribonucleotides or analogs thereof. Polynucleotides can have any three-dimensional structure and may perform any function. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

A “gene” refers to a polynucleotide containing at least one open reading frame (ORF) that is capable of encoding a particular polypeptide or protein after being transcribed and translated. A polynucleotide sequence can be used to identify larger fragments or full-length coding sequences of the gene with which they are associated. Methods of isolating larger fragment sequences are known to those of skill in the art.

“Gene expression” or alternatively a “gene product” refers to the nucleic acids or amino acids (e.g., peptide or polypeptide) generated when a gene is transcribed and translated.

The term “polypeptide” is used interchangeably with the term “protein” and in its broadest sense refers to a compound of two or more subunit amino acids, amino acid analogs, or peptidomimetics. The subunits can be linked by peptide bonds. In another embodiment, the subunit may be linked by other bonds, e.g., ester, ether, etc.

As used herein the term “amino acid” refers to either natural and/or unnatural or synthetic amino acids, and both the D and L optical isomers, amino acid analogs, and peptidomimetics. A peptide of three or more amino acids is commonly called an oligopeptide if the peptide chain is short. If the peptide chain is long, the peptide is commonly called a polypeptide or a protein.

The term “isolated” means separated from constituents, cellular and otherwise, in which the polynucleotide, peptide, polypeptide, protein, antibody or fragment(s) thereof, are normally associated with in nature. For example, an isolated polynucleotide is separated from the 3′ and 5′ contiguous nucleotides with which it is normally associated within its native or natural environment, e.g., on the chromosome. As is apparent to those of skill in the art, a non-naturally occurring polynucleotide, peptide, polypeptide, protein, antibody, or fragment(s) thereof, does not require “isolation” to distinguish it from its naturally occurring counterpart. In addition, a “concentrated,” “separated” or “diluted” polynucleotide, peptide, polypeptide, protein, antibody or fragment(s) thereof, is distinguishable from its naturally occurring counterpart in that the concentration or number of molecules per volume is greater in a “concentrated” version or less than in a “separated” version than that of its naturally occurring counterpart. A polynucleotide, peptide, polypeptide, protein, antibody, or fragment(s) thereof, which differs from the naturally occurring counterpart in its primary sequence or, for example, by its glycosylation pattern, need not be present in its isolated form since it is distinguishable from its naturally occurring counterpart by its primary sequence or, alternatively, by another characteristic such as glycosylation pattern. Thus, a non-naturally occurring polynucleotide is provided as a separate embodiment from the isolated naturally occurring polynucleotide. A protein produced in a bacterial cell is provided as a separate embodiment from the naturally occurring protein isolated from a eukaryotic cell in which it is produced in nature.

A “probe” when used in the context of polynucleotide manipulation refers to an oligonucleotide that is provided as a reagent to detect a target potentially present in a sample of interest by hybridizing with the target. Usually, a probe will comprise a label or a means by which a label can be attached, either before or subsequent to the hybridization reaction. Suitable labels include, but are not limited to radioisotopes, fluorochromes, chemiluminescent compounds, dyes, and proteins, including enzymes.

A “primer” is a short polynucleotide, generally with a free 3′-OH group that binds to a target or “template” potentially present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or a “set of primers” consisting of an “upstream” and a “downstream” primer, and a catalyst of polymerization, such as a DNA polymerase, and typically a thermally-stable polymerase enzyme. Methods for PCR are well known in the art, and taught, for example in PCR: A Practical Approach, M. MacPherson et al., IRL Press at Oxford University Press (1991). All processes of producing replicate copies of a polynucleotide, such as PCR or gene cloning, are collectively referred to herein as “replication.” A primer can also be used as a probe in hybridization reactions, such as Southern or Northern blot analyses (Sambrook et al., Molecular Cloning: A Laboratory Manual, 2nd edition (1989)).

As used herein, “expression” refers to the process by which DNA is transcribed into mRNA and/or the process by which the transcribed mRNA is subsequently translated into peptides, polypeptides or proteins. If the polynucleotide is derived from genomic DNA, expression may include splicing of the mRNA in a eukaryotic cell.

“Differentially expressed” as applied to a gene, refers to the differential production of the mRNA transcribed and/or translated from the gene or the protein product encoded by the gene. A differentially expressed gene may be overexpressed or underexpressed as compared to the expression level of a normal or control cell. However, as used herein, overexpression is an increase in gene expression and generally is at least 1.25 fold or, alternatively, at least 1.5 fold or, alternatively, at least 2 fold, or alternatively, at least 3 fold or alternatively, at least 4 fold expression over that detected in a normal or control counterpart cell or tissue. As used herein, underexpression, is a reduction of gene expression and generally is at least 1.25 fold, or alternatively, at least 1.5 fold, or alternatively, at least 2 fold or alternatively, at least 3 fold or alternatively, at least 4 fold expression under that detected in a normal or control counterpart cell or tissue. The term “differentially expressed” also refers to where expression in a cancer cell or cancerous tissue is detected but expression in a control cell or normal tissue (e.g. non-cancerous cell or tissue) is undetectable.

A high expression level of the gene may occur because of over expression of the gene or an increase in gene copy number. The gene may also be translated into increased protein levels because of deregulation or absence of a negative regulator.

The term “cDNA” refers to complementary DNA, i.e. mRNA molecules present in a cell or organism made into cDNA with an enzyme such as reverse transcriptase. A “cDNA library” is a collection of all of the mRNA molecules present in a cell or organism, all turned into cDNA molecules with the enzyme reverse transcriptase, then inserted into “vectors” (other DNA molecules that can continue to replicate after addition of foreign DNA). Exemplary vectors for libraries include bacteriophage (also known as “phage”), viruses that infect bacteria, for example, lambda phage. The library can then be probed for the specific cDNA (and thus mRNA) of interest.

As used herein, “solid phase support” or “solid support,” used interchangeably, is not limited to a specific type of support. Rather a large number of supports are available and are known to one of ordinary skill in the art. Solid phase supports include silica gels, resins, derivatized plastic films, glass beads, plastic beads, alumina gels, microarrays, and chips. As used herein, “solid support” also includes synthetic antigen-presenting matrices, cells, and liposomes. A suitable solid phase support may be selected on the basis of desired end use and suitability for various protocols. For example, for peptide synthesis, solid phase support may refer to resins such as polystyrene (e.g., PAM-resin obtained from Bachem Inc., Peninsula Laboratories), polyHIPE(R)™ resin (obtained from Aminotech, Canada), polyamide resin (obtained from Peninsula Laboratories), polystyrene resin grafted with polyethylene glycol (TentaGelR™, Rapp Polymere, Tubingen, Germany), or polydimethylacrylamide resin (obtained from Milligen/Biosearch, California).

A polynucleotide also can be attached to a solid support for use in high throughput screening assays. PCT WO 97/10365, for example, discloses the construction of high density oligonucleotide chips. See also, U.S. Pat. Nos. 5,405,783; 5,412,087 and 5,445,934. Using this method, the probes are synthesized on a derivatized glass surface to form chip arrays. Photoprotected nucleoside phosphoramidites are coupled to the glass surface, selectively deprotected by photolysis through a photolithographic mask and reacted with a second protected nucleoside phosphoramidite. The coupling/deprotection process is repeated until the desired probe is complete.

As an example, transcriptional activity can be assessed by measuring levels of messenger RNA using a gene chip such as the Affymetrix® HG-U133-Plus-2 GeneChips (Affmetrix, Santa Clara Calif.). High-throughput, real-time quantitation of RNA of a large number of genes of interest thus becomes possible in a reproducible system.

The terms “stringent hybridization conditions” refers to conditions under which a nucleic acid probe will specifically hybridize to its target subsequence, and to no other sequences. The conditions determining the stringency of hybridization include: temperature, ionic strength, and the concentration of denaturing agents such as formamide. Varying one of these factors may influence another factor and one of skill in the art will appreciate changes in the conditions to maintain the desired level of stringency. An example of a highly stringent hybridization is: 0.015M sodium chloride, 0.0015M sodium citrate at 65-68° C. or 0.015M sodium chloride, 0.0015M sodium citrate, and 50% formamide at 42° C. (see Sambrook, supra). An example of a “moderately stringent” hybridization is the conditions of: 0.015M sodium chloride, 0.0015M sodium citrate at 50-65° C. or 0.015M sodium chloride, 0.0015M sodium citrate, and 20% formamide at 37-50° C. The moderately stringent conditions are used when a moderate amount of nucleic acid mismatch is desired. One of skill in the art will appreciate that washing is part of the hybridization conditions. For example, washing conditions can include 02.×-0.1×SSC/0.1% SDS and temperatures from 42-68° C., wherein increasing temperature increases the stringency of the wash conditions.

When hybridization occurs in an antiparallel configuration between two single-stranded polynucleotides, the reaction is called “annealing” and those polynucleotides are described as “complementary.” A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to form hydrogen bonding with each other, according to generally accepted base-pairing rules.

A polynucleotide or polynucleotide region (or a polypeptide or polypeptide region) has a certain percentage (for example, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Current Protocols in Molecular Biology, Ausubel et al., eds., (1987) Supplement 30, section 7.7.18, Table 7.7.1. Preferably, default parameters are used for alignment. A preferred alignment program is BLAST, using default parameters. In particular, preferred programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant.

The term “cell proliferative disorders” shall include dysregulation of normal physiological function characterized by abnormal cell growth and/or division or loss of function. Examples of “cell proliferative disorders” includes but is not limited to hyperplasia, neoplasia, metaplasia, and various autoimmune disorders, e.g., those characterized by the dysregulation of T cell apoptosis.

As used herein, the terms “neoplastic cells,” “neoplastic disease,” “neoplasia,” “tumor,” “tumor cells,” “cancer,” and “cancer cells,” (used interchangeably) refer to cells which exhibit relatively autonomous growth, so that they exhibit an aberrant growth phenotype characterized by a significant loss of control of cell proliferation (i.e., de-regulated cell division). Neoplastic cells can be malignant or benign. A metastatic cell or tissue means that the cell can invade and destroy neighboring body structures. Cancer can include without limitation: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

“Suppressing” tumor growth indicates a reduction in tumor cell growth when contacted with a chemotherapeutic compared to tumor growth without a chemotherapeutic agent. Tumor cell growth can be assessed by any means known in the art, including, but not limited to, measuring tumor size, determining whether tumor cells are proliferating using a 3H-thymidine incorporation assay, measuring glucose uptake by FDG-PET (fluorodeoxyglucose positron emission tomography) imaging, or counting tumor cells. “Suppressing” tumor cell growth means any or all of the following states: slowing, delaying and stopping tumor growth, as well as tumor shrinkage.

A “composition” is a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include pharmaceutical excipients and additives, for example; proteins, peptides, amino acids, lipids, and carbohydrates (e.g., sugars, including monosaccharides and oligosaccharides; derivatized sugars such as alditols, aldonic acids, esterified sugars and the like; and polysaccharides or sugar polymers), which can be present singly or in combination, comprising alone or in combination 1-99.99% by weight or volume. Carbohydrate excipients include, for example; monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

The term “carrier” further includes a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl-quadrature-cyclodextrin), polyethylene glycols, flavoring agents, antimicrobial agents, sweeteners, antioxidants, antistatic agents, surfactants (e.g., polysorbates such as TWEEN 20™ and TWEEN 80™), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives and any of the above noted carriers with the additional provisio that they be acceptable for use in vivo. For examples of carriers, stabilizers and adjuvants, see Remington's Pharmaceutical Science., 15th Ed. (Mack Publ. Co., Easton (1975) and in the Physician's Desk Reference, 52nd ed., Medical Economics, Montvale, N.J. (1998).

An “effective amount” is an amount sufficient to effect beneficial or desired results. An effective amount can be administered in one or more administrations, applications or dosages.

A “subject,” “individual” or “patient” is used interchangeably herein, which refers to a vertebrate, preferably a mammal, more preferably a human. Mammals include, but are not limited to, mice, simians, humans, farm animals, sport animals, and pets.

An “inhibitor” of EZH2 as used herein reduces the N-methytransferase activity of EZH2. This inhibition may include, for example, reducing the association of EZH2 and the histone before they are bound together, reducing the association of EZH2 and histone after they are bound together, or binding the EZH2 active site, thus reducing N-methytransferase activity. EZH2 inhibitors are useful in pharmaceutical compositions for human or veterinary use, e.g., in the treatment of tumors and/or cancerous cell growth. EZH2 inhibitor compounds are useful in treating, for example: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

### Detection of EZH2 Mutations

The detection of EZH2 mutations can be done by any number of ways, for example: DNA sequencing, PCR based methods, including RT-PCR, microarray analysis, Southern blotting, Northern blotting and dip stick analysis.

The polymerase chain reaction (PCR) can be used to amplify and identify EZH2 mutations from either genomic DNA or RNA extracted from tumor tissue. PCR is well known in the art and is described in detail in Saiki et al., Science 1988, 239:487 and in U.S. Pat. No. 4,683,195 and U.S. Pat. No. 4,683,203.

Methods of detecting EZH2 mutations by hybridization are provided. The method comprises identifying a EZH2 mutation in a sample by contacting nucleic acid from the sample with a nucleic acid probe that is capable of hybridizing to nucleic acid with a EZH2 mutation or fragment thereof and detecting the hybridization. The nucleic acid probe is detectably labeled with a label such as a radioisotope, a fluorescent agent or a chromogenic agent. Radioisotopes can include without limitation; ³H, ³²P, ³³P and ³⁵S etc. Fluorescent agents can include without limitation: fluorescein, texas red, rhodamine, etc.

The probe used in detection that is capable of hybridizing to nucleic acid with a EZH2 mutation can be from about 8 nucleotides to about 100 nucleotides, from about 10 nucleotides to about 75 nucleotides, from about 15 nucleotides to about 50 nucleotides, or about 20 to about 30 nucleotides. The probe or probes can be provided in a kit, which comprise at least one oligonucleotide probe that hybridizes to or hybridizes adjacent to a EZH2 mutation. The kit can also provide instructions for analysis of patient cancer samples that can contain a EZH2 mutation.

Single stranded conformational polymorphism (SSCP) can also be used to detect EZH2 mutations. This technique is well described in Orita et al., PNAS 1989, 86:2766-2770.

Antibodies directed against EZH2 can be useful in the detection of mutated forms of EZH2. Antibodies can be generated which recognize and specifically bind only a specific mutant form of EZH2 and do not bind (or weakly bind) to wild type EZH2. These antibodies would be useful in determining which specific mutation was present and also in quantifying the level of EZH2 protein. For example, an antibody can be directed against the change in amino acid at tyrosine 641 (Y641). Such antibodies can be generated by using peptides containing an EZH2 mutation.

### Measurement of Gene Expression

Detection of gene expression can be by any appropriate method, including for example, detecting the quantity of mRNA transcribed from the gene or the quantity of cDNA produced from the reverse transcription of the mRNA transcribed from the gene or the quantity of the polypeptide or protein encoded by the gene. These methods can be performed on a sample by sample basis or modified for high throughput analysis. For example, using Affymetrix™ U133 microarray chips (Affymax, Santa Clara, Calif.).

In one aspect, gene expression is detected and quantitated by hybridization to a probe that specifically hybridizes to the appropriate probe for that biomarker. The probes also can be attached to a solid support for use in high throughput screening assays using methods known in the art. WO 97/10365 and U.S. Pat. Nos. 5,405,783, 5,412,087 and 5,445,934, for example, disclose the construction of high density oligonucleotide chips which can contain one or more of the sequences disclosed herein. Using the methods disclosed in U.S. Pat. Nos. 5,405,783, 5,412,087 and 5,445,934, the probes of this invention are synthesized on a derivatized glass surface. Photoprotected nucleoside phosphoramidites are coupled to the glass surface, selectively deprotected by photolysis through a photolithographic mask, and reacted with a second protected nucleoside phosphoramidite. The coupling/deprotection process is repeated until the desired probe is complete.

In one aspect, the expression level of a gene is determined through exposure of a nucleic acid sample to the probe-modified chip. Extracted nucleic acid is labeled, for example, with a fluorescent tag, preferably during an amplification step. Hybridization of the labeled sample is performed at an appropriate stringency level. The degree of probe-nucleic acid hybridization is quantitatively measured using a detection device. See U.S. Pat. Nos. 5,578,832 and 5,631,734.

Alternatively any one of gene copy number, transcription, or translation can be determined using known techniques. For example, an amplification method such as PCR may be useful. General procedures for PCR are taught in MacPherson et al., PCR: A Practical Approach, (IRL Press at Oxford University Press (1991)). However, PCR conditions used for each application reaction are empirically determined. A number of parameters influence the success of a reaction. Among them are annealing temperature and time, extension time, Mg 2+ and/or ATP concentration, pH, and the relative concentration of primers, templates, and deoxyribonucleotides. After amplification, the resulting DNA fragments can be detected by agarose gel electrophoresis followed by visualization with ethidium bromide staining and ultraviolet illumination.

In one embodiment, the hybridized nucleic acids are detected by detecting one or more labels attached to the sample nucleic acids. The labels can be incorporated by any of a number of means well known to those of skill in the art. However, in one aspect, the label is simultaneously incorporated during the amplification step in the preparation of the sample nucleic acid. Thus, for example, polymerase chain reaction (PCR) with labeled primers or labeled nucleotides will provide a labeled amplification product. In a separate embodiment, transcription amplification, as described above, using a labeled nucleotide (e.g. fluorescein-labeled UTP and/or CTP) incorporates a label in to the transcribed nucleic acids.

Alternatively, a label may be added directly to the original nucleic acid sample (e.g., mRNA, polyA, mRNA, cDNA, etc.) or to the amplification product after the amplification is completed. Means of attaching labels to nucleic acids are well known to those of skill in the art and include, for example nick translation or end-labeling (e.g. with a labeled RNA) by kinasing of the nucleic acid and subsequent attachment (ligation) of a nucleic acid linker joining the sample nucleic acid to a label (e.g., a fluorophore).

Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™ Life Technologies, Grand Island, N.Y.), fluorescent dyes (e.g., fluorescein, texas red, rhodamine, green fluorescent protein, and the like), radiolabels (e.g., 3^{H}, 125^{I}, 35^{S}, 14^{C}, or 32^{P}) enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

Detection of labels is well known to those of skill in the art. Thus, for example, radiolabels may be detected using photographic film or scintillation counters, fluorescent markers may be detected using a photodetector to detect emitted light. Enzymatic labels are typically detected by providing the enzyme with a substrate and detecting the reaction product produced by the action of the enzyme on the substrate, and calorimetric labels are detected by simply visualizing the coloured label.

The detectable label may be added to the target (sample) nucleic acid(s) prior to, or after the hybridization, such as described in WO 97/10365. These detectable labels are directly attached to or incorporated into the target (sample) nucleic acid prior to hybridization. In contrast, “indirect labels” are joined to the hybrid duplex after hybridization. Generally, the indirect label is attached to a binding moiety that has been attached to the target nucleic acid prior to the hybridization. For example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization with Nucleic Acid Probes, P. Tijssen, ed. Elsevier, N.Y. (1993).

### Detection of Polypeptides

An EZH2 mutation when translated into protein can be detected by specific antibodies. A mutation in an EZH2 protein can change the antigenicity, so that an antibody raised against an EZH2 mutant antigen (e.g. a specific peptide containing a mutation) will specifically bind the mutant EZH2 and not recognize the wild-type.

Expression level of an EZH2 mutant can also be determined by examining protein expression. Determining the protein level involves measuring the amount of any immunospecific binding that occurs between an antibody that selectively recognizes and binds to the polypeptide of the biomarker in a sample obtained from a patient and comparing this to the amount of immunospecific binding of at least one biomarker in a control sample. The amount of protein expression of an EZH2 mutant protein can be increased or reduced when compared with control expression. A variety of techniques are available in the art for protein analysis. They include but are not limited to radioimmunoassays, ELISA (enzyme linked immunosorbent assays), “sandwich” immunoassays, immunoradiometric assays, in situ immunoassays (using e.g., colloidal gold, enzyme or radioisotope labels), western blot analysis, immunoprecipitation assays, immunofluorescent assays, flow cytometry, immunohistochemistry, confocal microscopy, enzymatic assays, surface plasmon resonance and PAGE-SDS.

### Assaying for Biomarkers

Once a patient has been assayed to have a specific EZH2 MCS, administration of an EZH2 inhibitor to a patient can be affected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents may be empirically adjusted.

Compound EI1 is a member of the pyridinonyl substituted indolines class of compounds and is a SAM competitive class of highly specific inhibitors of EZH2. The chemical name of EI1 is 6-cyano-N-((4,6-dimethyl-2-oxo-1,2-dihydropyridin-3-yl)methyl)-1-(pentan-3-yl)-1H-indole-4-carboxamide and is published in Qi et al., Proc. Natl. Acad. Sci. USA 2012; 109(52):21360-21365. EI1 has a molecular weight of 390.8, and is SAM competitive. It has an IC50 of 9.4 nM for wild type EZH2 and an IC50 of 13.2 nM for Y641F EZH2.

### EI1

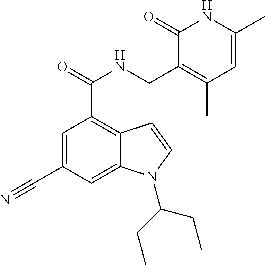 [SMILES: CCC(CC)n1ccc2c(C(=O)NCc3c(C)cc(C)[nH]c3=O)cc(C#N)cc21]

The EZH2 MCS can be assayed for after EZH2 inhibitor administration in order to determine if the chemotherapeutic treatment remains appropriate. In addition, the MCS can be assayed for in multiple timepoints after a single chemotherapeutic administration. For example, after an initial bolus of a chemotherapeutic is administered, the EZH2 MCS can be assayed for at 1 hour, 2 hours, 3 hours, 4 hours, 8 hours, 16 hours, 24 hours, 48 hours, 3 days, 1 week or 1 month or several months after the first treatment.

The EZH2 MCS can be assayed for after each chemotherapeutic administration, so if there are multiple chemotherapeutic administrations, then assaying for a histone profile after each administration can determine continued course of treatment. The patient could undergo multiple chemotherapeutic administrations and the MCS assayed at different time points. For example, a course of treatment may require administration of an initial dose of chemotherapeutic, a second dose a specified time period later, and still a third dose. An MCS could be assayed for at 1 hour, 2 hours, 3 hours, 4 hours, 8 hours, 16 hours, 24 hours, 48 hours, 3 days, 1 week or 1 month or several months after administration of each dose of chemotherapeutic.

Finally, there is administration of different chemotherapeutics, followed by assaying for an EZH2 MCS. In this embodiment, more than one chemotherapeutic is chosen and administered to the patient. An EZH2 MCS can then be assayed for after administration of each different chemotherapeutic. This assay can also be done at multiple time points after administration of the different chemotherapeutics. For example, a first chemotherapeutic could be administered to the patient and an EZH2 MCS assayed for at 1 hour, 2 hours, 3 hours, 4 hours, 8 hours, 16 hours, 24 hours, 48 hours, 3 days, 1 week or 1 month or several months after administration. A second chemotherapeutic could then be administered and an EZH2 MCS could be assayed for again at 1 hour, 2 hours, 3 hours, 4 hours, 8 hours, 16 hours, 24 hours, 48 hours, 3 days, 1 week or 1 month or several months after administration of the second chemotherapeutic.

Kits for assessing an EZH2 MCS can be made. For example, a kit comprising reagents for EZH2 MCS can be used in conjunction with mass spectrometry for performing the histone profiling.

### Screening for EZH2 Inhibitors

It is possible to use MCS profiles to screen for EZH2 inhibitors. This method comprises choosing or engineering a cell with an EZH2 gain of function mutation that results in increased H3K27me3, the cell is then contacted with the candidate EZH2 inhibitor compound and the contacted cell is assayed. As the EZH2 mutation shows increased methyltransferase activity, assaying for a reduction in methyltransferase activity and a reduction in the methylation at H3K27me3 when compared to a control cell would indicate that the candidate compound is an EZH2 inhibitor. Alternatively, the contacted cell is assayed for reduction in proliferation or increase in apoptosis. A reduction in proliferation or increase in apoptosis over untreated control sample is a positive result, indicating that the candidate compound inhibits EZH2.

### EXAMPLES

### Example 1

### MCS by Mass Spectrometry

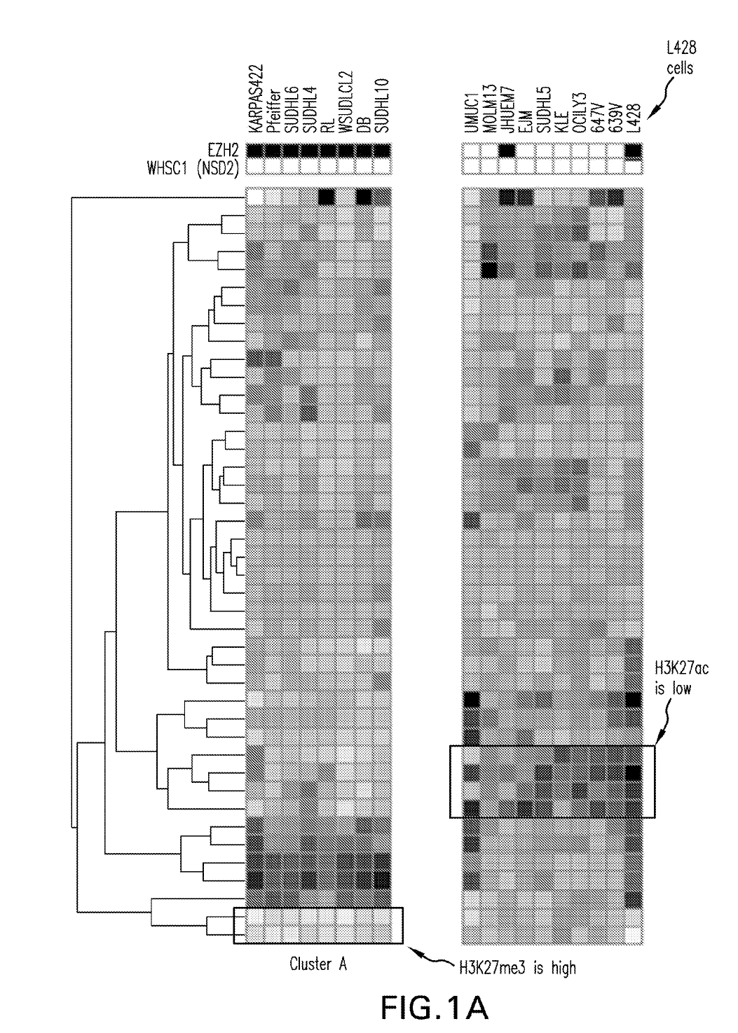

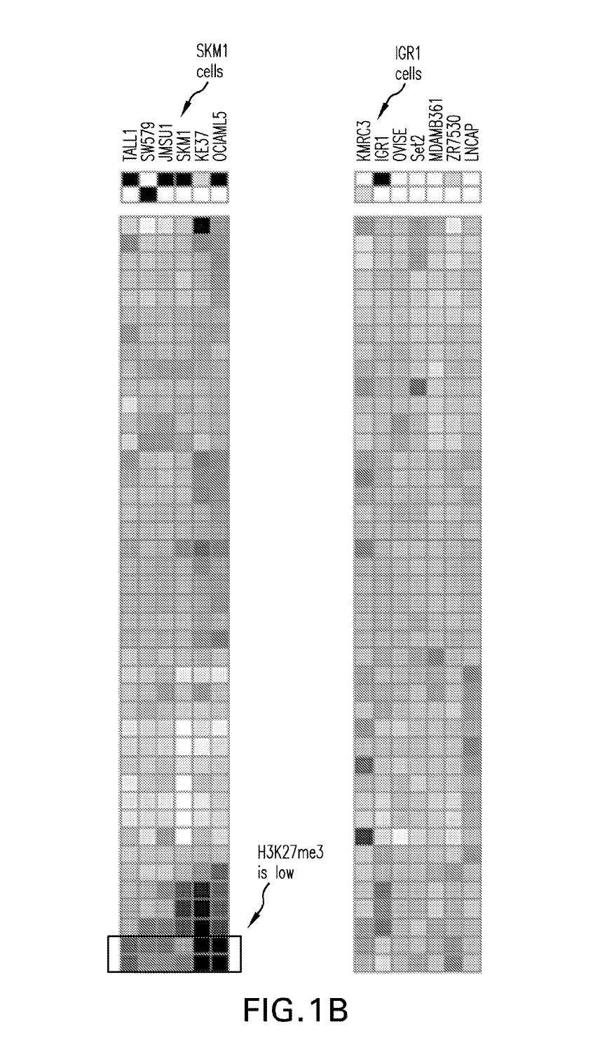

FIG. 1 shows a heat map of EZH2 histone methylation patterns, and how EZH2 mutations fall into different MCS based on specific histone methylation profiles, for example when H3K27me3 is low or high.

HeLa, K562, and 293T cells were acquired from ATCC (CCL-2, CCL-243 and CRL-11268 respectively, Manassas, Va.). HeLa cell media consisted of RPMI without lysine, arginine, or methionine (Caisson Labs, North Logan, Utah), supplemented with 5% dialyzed fetal bovine serum (dFBS), 1% penicillin-streptomycin-glutamine (PSG) (Life Technologies, Grand Island, N.Y.), 30 mg/L L-methionine, 80 mg/L (¹³C₆, ¹⁵N₄)-L-arginine, 40 mg/L L-lysine. K562 media consisted of RPMI without lysine, arginine, or methionine, supplemented with 10% dFBS, 1% PSG, 30 mg/L L-methionine, 80 mg/L (¹³C₆, ¹⁵N₄)-L-arginine, 40 mg/L L-lysine plus 200 mg/L L-proline. 293T media consisted of DMEM without lysine, arginine, or methionine (Caisson Labs), supplemented with 10% dFBS, 4.5 g/L glucose, 1% PSG, 56 mg/L (¹³C₆, ¹⁵N₄)-L-arginine, 146 mg/L L-lysine, 30 mg/L L-methionine plus 200 mg/L L-proline. Lines were propagated according to standard tissue culture practices. At time of harvest, cells were washed twice with phosphate buffered saline (Life Technologies, Grand Island, N.Y.), pelleted at 1,000×g for 2 minutes, the supernatant removed, and the cell pellet flash frozen in liquid nitrogen to be stored at −80° C. until thawed for histone preparation. These lines are referred to as “R₁₀” standard cell lines to indicate that arginines in the proteins in these cells are labeled using stable isotopes to an additional 10 Daltons. All chemicals are from Sigma-Aldrich (St. Louis, Mo.) unless otherwise noted.

### Histone Extraction and Preparation for Analysis

Frozen cell pellets from sample and R₁₀ standard cell lines were thawed on ice. Cells were resuspended in 1 mL lysis buffer (250 mM sucrose, 60 mM KCl, 15 mM NaCl, 15 mM Tris pH 7.5 (Life Technologies, Grand Island, N.Y.), 5 mM MgCl₂, 1 mM CaCl₂, 1 mM DTT (Thermo Scientific, Waltham, Mass.), 10 mM sodium butyrate, 0.5 mM AEBSF (Merck, Darmstadt, Germany), 5 nM Microcystin LR (Merck), 0.3% NP-40 substitute), pelleted at 10,000×g for 1 min, and the supernatant discarded. This operation was repeated a total of 3 times for each cell pellet. The resulting nucleus pellet was resuspended in 0.4 N H₂SO₄ and incubated for 4 hours at room temperature with gentle orbital shaking. Subsequently, the samples were pelleted at 10,000×g for 5 min, and the supernatant removed to a new tube. The supernatant was then brought up to 20% trichloroacetic acid (using a 60% stock solution) and incubated for 30 min on ice. Samples were spun at 20,000×g for 15 minutes, the supernatant discarded, and the pellet gently washed with acetone and air dried for 15 minutes. The resulting histones were resuspended in 50 μl HPLC-grade water, and the protein concentration was measured using the Coomassie Plus® Protein Assay (Thermo Scientific, Waltham Mass.).

Samples were derivatized in a manner highly similar to Garcia's method (Garcia et al., Nat. Protoc. 2007; 2:933-938). The R₁₀ standard mix of histones consisted of equal parts (by protein concentration) of R₁₀-labeled HeLa, K562, and 293T histones. 25 μg of sample histone was mixed with 25 μg of the R₁₀ standard mix of histones and brought up to 100 mM sodium phosphate buffer pH 8.0 in a total volume of 65 μl. 195 μl of 400 mM NHS-propionate (synthesized in-house) in anhydrous MeOH was added via Bravo liquid handling system (Agilent Technologies, Santa Clara, Calif.) with tip mixing for 30 minutes. Samples were desalted on an Oasis® 30 mg HLB solid phase extraction plate (Waters, Milford, Mass.). Samples were loaded and washed at 0.1% trifluoroacetic acid (TFA), 20% acetonitrile (ACN) and eluted at 0.1% TFA, 60% ACN. The eluates were vacuum concentrated to dryness. Samples were proteolytically digested using 1 μg of sequencing grade modified trypsin (Promega, Madison Wis.) per sample in 100 μL 50 mM ammonium bicarbonate at 37° C. Samples were then vacuum concentrated to dryness again. Samples were resuspended in 100 μl of 100 mM NHS propionate in anhydrous MeOH, plus 11 μL 20 mM phosphate buffer pH 8.0 and incubated for 1 hour with tip mixing. After being vacuum concentrated to dryness, samples were desalted on a SepPak® 100 mg C18 solid phase extraction plate (Waters, Milford, Mass.). Samples were loaded and washed at 0.1% TFA and eluted at 0.1% TFA, 50% ACN. Eluates were once more vacuum concentrated to dryness before being resuspended in 50 μL 3% ACN 5% formic acid (FA) and diluted 1:10 prior to data acquisition.

### Targeted LCMS Data Acquisition

Samples were chromatographically separated using a Proxeon Easy NanoLC 1000® (Thermo Scientific, Waltham Mass.) fitted with a PicoFrit 75 μm inner diameter capillary packed in-house with 200 mm of C₁₈ Reprosil® beads (1.9 μm particle size, 200 Å pore size). The column was heated to 50° C. during separation. Buffer A consisted of 0.1% FA, 3% ACN and Buffer B consisted of 0.1% FA, 90% ACN. Samples were loaded in Buffer A and eluted with a linear gradient from 3-40% of Buffer B over 45 minutes, 40-90% Buffer B over 5 minutes, and then held at 90% Buffer B for 10 minutes at 200 nL/min.

Eluting peptides were introduced into a Q-Exactive® mass spectrometer (Thermo Scientific, Waltham Mass.) via nanoelectrospray at 2.15 kV. A full scan MS was acquired at a resolution of 35,000 from 300 to 1800 m/z. Each full scan was followed by up to 17 scheduled, targeted HCD MS/MS scans. Each targeted peptide species was subjected to targeting for three to twenty minutes, depending empirical chromatographic properties, centered on the average observed retention time of two scheduling runs containing synthetic peptides for many possible peptide/modification combinations on histone H3. See Table 1 below for an example, including normalized HCD collision energies for each species. Table 2 provides standard nomenclature and biochemical abbreviations for the peptide species that were monitored.

### Data Analysis

Files were imported into Skyline software with targets for many possible peptide/modification combinations on H3 (Skyline software available at University of Washington School of Medicine, Seattle, Wash.). Transitions were chosen based on selectivity for the given modification or modification combinations and detectability. Each sample and modification was manually validated using the criteria of retention time agreement with other samples and the co-eluting presence of all transitions. Heavy-to-light ratios were extracted based on transition area integration using Skyline defaults. All ratios were normalized to the heavy-to-light ratio of the H3 41-49 peptide and log₂ transformed. Data for each modification were normalized by the median of all samples before clustering. Clustering was performed in Gene-E using unsupervised hierarchical methods with the following methods: Euclidean distance metric, complete linkage, row and column clustering.

TABLE 1

| | Start | End | Normalized HCD | Charge | |
|---|---|---|---|---|---|
| Mass | time | time | Collision Energy | State | |
| [m/z] | [min] | [min] | (%) | [z] | Targeted Species* |
| 394.7349 | 0.0 | 20.0 | 39 | 2 | H3K4me2-L |
| 399.7390 | 0.0 | 20.0 | 39 | 2 | H3K4me2-H |
| 401.2426 | 21.9 | 24.9 | 39 | 4 | H3K27me2K36me2-L |
| 401.7245 | 20.1 | 25.1 | 21 | 2 | H3K4ac1-L |
| 401.7427 | 0.0 | 20.0 | 39 | 2 | H3K4me3-L |
| 403.7447 | 21.9 | 24.9 | 39 | 4 | H3K27me2K36me2-H |
| 404.7465 | 21.9 | 24.9 | 39 | 4 | H3K27me3K36me2-L + |
| | | | | | H3K27me2K36me3-L |
| 406.7286 | 20.1 | 25.1 | 21 | 2 | H3K4ac1-H |
| 406.7468 | 0.0 | 20.0 | 39 | 2 | H3K4me3-H |
| 407.2486 | 21.9 | 24.9 | 39 | 4 | H3K27me3K36me2-H + |
| | | | | | H3K27me2K36me3-H |
| 408.2504 | 21.8 | 24.8 | 39 | 4 | H3K27me3K36me3-L |
| 408.7323 | 22.7 | 25.7 | 21 | 2 | H3K4me0-L |
| 410.7525 | 21.8 | 24.8 | 39 | 4 | H3K27me3K36me3-H |
| 413.7365 | 22.7 | 25.7 | 21 | 2 | H3K4me0-H |
| 415.7402 | 25.1 | 28.8 | 21 | 2 | H3K4me1-L |
| 420.7443 | 25.1 | 28.8 | 21 | 2 | H3K4me1-H |
| 514.2984 | 20.1 | 23.1 | 35 | 2 | H3K9me2K14ac1-L |
| 519.3025 | 20.1 | 23.1 | 35 | 2 | H3K9me2K14ac1-H |
| 521.2880 | 21.3 | 29.8 | 25 | 2 | H3K9ac1K14ac1-L |
| 521.3062 | 19.9 | 22.9 | 33 | 2 | H3K9me3K14ac1-L |
| 521.3062 | 20.9 | 23.9 | 33 | 2 | H3K9me2K14ac0-L |
| 526.2921 | 21.3 | 29.8 | 25 | 2 | H3K9ac1K14ac1-H |
| 526.3103 | 19.9 | 22.9 | 33 | 2 | H3K9me3K14ac1-H |
| 526.3103 | 20.9 | 23.9 | 33 | 2 | H3K9me2K14ac0-H |
| 528.2958 | 25.6 | 28.6 | 25 | 2 | H3K9ac1K14ac0-L + |
| | | | | | H3K9me0K14ac1-L |
| 528.3140 | 20.8 | 23.8 | 35 | 2 | H3K9me3K14ac0-L |
| 533.3000 | 25.6 | 28.6 | 25 | 2 | H3K9ac1K14ac0-H + |
| | | | | | H3K9me0K14ac1-H |
| 533.3182 | 20.8 | 23.8 | 35 | 2 | H3K9me3K14ac0-H |
| 535.3037 | 26.9 | 31.2 | 23 | 2 | H3K9me0K14ac0-L |
| 535.3037 | 29.0 | 32.0 | 23 | 2 | H3K9me1K14ac1-L |
| 539.3141 | 24.8 | 27.8 | 31 | 3 | H3K27ac1K36me2-L |
| 540.3078 | 26.9 | 31.2 | 23 | 2 | H3K9me0K14ac0-H |
| 540.3078 | 29.0 | 32.0 | 23 | 2 | H3K9me1K14ac1-H |
| 542.3115 | 30.8 | 33.8 | 23 | 2 | H3K9me1K14ac0-L |
| 542.6502 | 24.8 | 27.8 | 31 | 3 | H3K27ac1K36me2-H |
| 543.9860 | 24.8 | 27.8 | 23 | 3 | H3K27ac1K36me3-L |
| 543.9860 | 24.4 | 27.8 | 35 | 3 | H3K27me2K36me0-L |
| 543.9860 | 25.6 | 29.0 | 35 | 3 | H3K27me0K36me2-L |
| 544.8142 | 30.4 | 33.4 | 31 | 2 | H3(41-49)-L |
| 547.3156 | 30.8 | 33.8 | 23 | 2 | H3K9me1K14ac0-H |
| 547.3221 | 24.4 | 27.8 | 35 | 3 | H3K27me2K36me0-H |
| 547.3221 | 25.6 | 29.0 | 35 | 3 | H3K27me0K36me2-H |
| 547.3221 | 24.8 | 27.8 | 23 | 3 | H3K27ac1K36me3-H |
| 548.6458 | 29.6 | 32.6 | 25 | 3 | H3K27ac1K36me0-L |
| 548.6579 | 28.3 | 31.3 | 31 | 3 | H3K27me1K36me2-L |
| 548.6579 | 24.0 | 29.7 | 31 | 3 | H3K27me0K36me3-L |
| 548.6579 | 26.2 | 29.2 | 31 | 3 | H3K27me2K36me1-L |
| 548.6579 | 23.2 | 28.9 | 31 | 3 | H3K27me3K36me0-L |
| 551.9819 | 29.6 | 32.6 | 25 | 3 | H3K27ac1K36me0-H |
| 551.9940 | 28.3 | 31.3 | 31 | 3 | H3K27me1K36me2-H |
| 551.9940 | 23.2 | 28.9 | 31 | 3 | H3K27me3K36me0-H |
| 551.9940 | 26.2 | 29.2 | 31 | 3 | H3K27me2K36me1-H |
| 551.9940 | 24.0 | 29.7 | 31 | 3 | H3K27me0K36me3-H |
| 553.3177 | 30.8 | 34.8 | 25 | 3 | H3K27me0K36me0-L + |
| | | | | | H3K27ac1K36me1-L |
| 553.3298 | 28.2 | 31.2 | 33 | 3 | H3K27me1K36me3-L |
| 553.3298 | 25.3 | 29.9 | 33 | 3 | H3K27me3K36me1-L |
| 554.2815 | 21.5 | 24.5 | 35 | 2 | H3K9me2S10ph1K14ac1-L |
| 554.8225 | 30.4 | 33.4 | 31 | 2 | H3(41-49)-H |
| 556.6537 | 30.8 | 34.8 | 25 | 3 | H3K27ac1K36me1-H + |
| | | | | | H3K27me0K36me0-H |
| 556.6659 | 28.2 | 31.2 | 33 | 3 | H3K27me1K36me3-H |
| 556.6659 | 25.3 | 29.9 | 33 | 3 | H3K27me3K36me1-H |
| 557.9895 | 33.5 | 36.5 | 25 | 3 | H3K27me1K36me0-L |
| 557.9895 | 32.4 | 35.4 | 25 | 3 | H3K27me0K36me1-L |
| 558.6493 | 30.6 | 33.6 | 25 | 3 | H3.3K27me0K36me0-L |
| 559.2857 | 21.5 | 24.5 | 35 | 2 | H3K9me2S10ph1K14ac1-H |
| 561.2712 | 25.9 | 28.9 | 25 | 2 | H3K9ac1S10ph1K14ac1-L |
| 561.2894 | 21.3 | 24.3 | 33 | 2 | H3K9me3S10ph1K14ac1-L |
| 561.2894 | 22.6 | 25.6 | 33 | 2 | H3K9me2S10ph1K14ac0-L |
| 561.3256 | 33.5 | 36.5 | 25 | 3 | H3K27me1K36me0-H |
| 561.3256 | 32.4 | 35.4 | 25 | 3 | H3K27me0K36me1-H |
| 561.9854 | 30.6 | 33.6 | 25 | 3 | H3.3K27me0K36me0-H |
| 562.6614 | 35.0 | 38.0 | 25 | 3 | H3K27me1K36me1-L |
| 563.8326 | 35.5 | 38.5 | 25 | 2 | H3K18ac1K23ac1-L |
| 565.9975 | 35.0 | 38.0 | 25 | 3 | H3K27me1K36me1-H |
| 566.2753 | 25.9 | 28.9 | 25 | 2 | H3K9ac1S10ph1K14ac1-H |
| 566.2935 | 21.3 | 24.3 | 33 | 2 | H3K9me3S10ph1K14ac1-H |
| 566.2935 | 22.6 | 25.6 | 33 | 2 | H3K9me2S10ph1K14ac0-H |
| 568.2790 | 27.9 | 30.9 | 25 | 2 | H3K9ac1S10ph1K14ac0-L + |
| | | | | | H3K9me0S10ph1K14ac1-L |
| 568.2972 | 22.4 | 25.4 | 35 | 2 | H3K9me3S10ph1K14ac0-L |
| 568.8367 | 35.5 | 38.5 | 25 | 2 | H3K18ac1K23ac1-H |
| 570.8404 | 37.2 | 40.3 | 24 | 2 | H3K18ac1K23ac0-L + |
| | | | | | H3K18ac0K23ac1-L |
| 573.2831 | 27.9 | 30.9 | 25 | 2 | H3K9ac1S10ph1K14ac0-H + |
| | | | | | H3K9me0S10ph1K14ac1-H |
| 573.3013 | 22.4 | 25.4 | 35 | 2 | H3K9me3S10ph1K14ac0-H |
| 575.2868 | 30.0 | 33.0 | 23 | 2 | H3K9me0S10ph1K14ac0-L |
| 575.2868 | 31.5 | 34.5 | 23 | 2 | H3K9me1S10ph1K14ac1-L |
| 575.8445 | 37.2 | 40.3 | 24 | 2 | H3K18ac1K23ac0-H + |
| | | | | | H3K18ac0K23ac1-H |
| 577.8482 | 38.9 | 41.9 | 25 | 2 | H3K18ac0K23ac0-L |
| 580.2910 | 31.5 | 34.5 | 23 | 2 | H3K9me1S10ph1K14ac1-H |
| 580.2910 | 30.0 | 33.0 | 23 | 2 | H3K9me0S10ph1K14ac0-H |
| 582.2946 | 33.4 | 36.4 | 23 | 2 | H3K9me1S10ph1K14ac0-L |
| 582.8524 | 38.9 | 41.9 | 25 | 2 | H3K18ac0K23ac0-H |
| 587.2988 | 33.4 | 36.4 | 23 | 2 | H3K9me1S10ph1K14ac0-H |
| 634.8697 | 37.9 | 40.9 | 25 | 2 | H3K18ub1K23ac0-L + |
| | | | | | H3K18ac0K23ub1-L |
| 639.8738 | 37.9 | 40.9 | 25 | 2 | H3K18ub1K23ac0-H + |
| | | | | | H3K18ac0K23ub1-H |
| 667.8876 | 36.9 | 39.9 | 35 | 2 | H3K56me2-L |
| 672.8917 | 36.9 | 39.9 | 35 | 2 | H3K56me2-H |
| 681.8850 | 43.4 | 46.4 | 25 | 2 | H3K56me0-L |
| 686.8891 | 43.4 | 46.4 | 25 | 2 | H3K56me0-H |
| 688.8928 | 43.4 | 47.2 | 25 | 2 | H3K56me1-L |
| 693.8970 | 43.4 | 47.2 | 25 | 2 | H3K56me1-H |
| 710.3775 | 38.5 | 41.5 | 35 | 2 | H3K79me2-L |
| 715.3817 | 38.5 | 41.5 | 35 | 2 | H3K79me2-H |
| 724.3750 | 45.5 | 48.5 | 25 | 2 | H3K79me0-L |
| 729.3791 | 45.5 | 48.5 | 25 | 2 | H3K79me0-H |
| 731.3828 | 46.4 | 50.8 | 25 | 2 | H3K79me1-L |
| 736.3870 | 46.4 | 50.8 | 25 | 2 | H3K79me1-H |

The label of the species targeted refers to the resultant derivatized peptide containing the stated modification after preparation as described in the Methods section. In some cases one set of (m/z, time window) coordinates may cover multiple species. “-L” denotes “light” peptides derived from the CCLE cell line while “-H” denotes “heavy” peptides derived from the R10 standard mix cell lines.

TABLE 2
Peptides monitored by Global Chromatin
Profiling with Histone Nomenclature
and Biochemical Abbreviations

| | Base | Modified |
|---|---|---|
| Nomenclature | Sequence | Sequence |
| H3K4me0 | TKQTAR | (pr)-T(Kpr)QTAR |
| | (SEQ ID | |
| | NO. 3) | |
| H3K4me1 | TKQTAR | (pr)-T(Kme1pr)QTAR |
| | (SEQ ID | |
| | NO. 3) | |
| H3K4me2 | TKQTAR | (pr)-T(Kme2)QTAR |
| | (SEQ ID | |
| | NO. 3) | |
| H3K4ac1 | TKQTAR | (pr)-T(Kac)QTAR |
| | (SEQ ID | |
| | NO. 3) | |
| H3K9me0K14ac0 | KSTGGKAPR | (pr)-(Kpr)STGG |
| | (SEQ ID | (Kpr)APR |
| | NO. 4) | |
| H3K9me0K14ac1 | KSTGGKAPR | (pr)-(Kpr)STGG |
| | (SEQ ID | (Kac)APR |
| | NO. 4) | |
| H3K9me1K14ac0 | KSTGGKAPR | (pr)-(Kme1pr)STGG |
| | (SEQ ID | (Kpr)APR |
| | NO. 4) | |
| H3K9me1K14ac1 | KSTGGKAPR | (pr)-(Kme1pr)STGG |
| | (SEQ ID | (Kac)APR |
| | NO. 4) | |
| H3K9me2K14ac0 | KSTGGKAPR | (pr)-(Kme2)STGG |
| | (SEQ ID | (Kpr)APR |
| | NO. 4) | |
| H3K9me2K14ac1 | KSTGGKAPR | (pr)-(Kme2)STGG |
| | (SEQ ID | (Kac)APR |
| | NO. 4) | |
| H3K9me3K14ac0 | KSTGGKAPR | (pr)-(Kme3)STGG |
| | (SEQ ID | (Kpr)APR |
| | NO. 4) | |
| H3K9me3K14ac1 | KSTGGKAPR | (pr)-(Kme3)STGG |
| | (SEQ ID | (Kac)APR |
| | NO. 4) | |
| H3K9ac1K14ac0 | KSTGGKAPR | (pr)-(Kac)STGG |
| | (SEQ ID | (Kpr)APR |
| | NO. 4) | |
| H3K9ac1K14ac1 | KSTGGKAPR | (pr)-(Kac)STGG |
| | (SEQ ID | (Kac)APR |
| | NO. 4) | |
| H3K18ac0K23ac0 | KQLATKAAR | (pr)-(Kpr)QLAT |
| | (SEQ ID | (Kpr)AAR |
| | NO. 5) | |
| H3K18ac1K23ac0 | KQLATKAAR | (pr)-(Kac)QLAT |
| | (SEQ ID | (Kpr)AAR |
| | NO. 5) | |
| H3K18ac0K23ac1 | KQLATKAAR | (pr)-(Kpr)QLAT |
| | (SEQ ID | (Kac)AAR |
| | NO. 5) | |
| H3K18ac1K23ac1 | KQLATKAAR | (pr)-(Kac)QLAT |
| | (SEQ ID | (Kac)AAR |
| | NO. 5) | |
| H3K18ac0K23ubq1 | KQLATKAAR | (pr)-(Kpr)QLAT |
| | (SEQ ID | (KGGpr)AAR |
| | NO. 5) | |
| H3K27me0K36me0 | KSAPATG | (pr)-(Kpr)SAPATGGV |
| | GVKKPHR | (Kpr)(Kpr)PHR |
| | (SEQ ID | |
| | NO. 6) | |
| H3K27me0K36me1 | KSAPATG | (pr)-(Kpr)SAPATGGV |
| | GVKKPHR | (Kme1pr)(Kpr)PHR |
| | (SEQ ID | |
| | NO. 6) | |
| H3K27me0K36me2 | KSAPATG | (pr)-(Kpr)SAPATGGV |
| | GVKKPHR | (Kme2)(Kpr)PHR |
| | (SEQ ID | |
| | NO. 6) | |
| H3K27me0K36me3 | KSAPATG | (pr)-(Kpr)SAPATGGV |
| | GVKKPHR | (Kme3)(Kpr)PHR |
| | (SEQ ID | |
| | NO. 6) | |
| H3K27me1K36me0 | KSAPATG | (pr)-(Kme1pr)SAPATGGV |
| | GVKKPHR | (Kpr)(Kpr)PHR |
| | (SEQ ID | |
| | NO. 6) | |
| H3K27me1K36me1 | KSAPATG | (pr)-(Kme1pr)SAPATGGV |
| | GVKKPHR | (Kme1pr)(Kpr)PHR |
| | (SEQ ID | |
| | NO. 6) | |
| H3K27me1K36me2 | KSAPATG | (pr)-(Kme1pr)SAPATGGV |
| | GVKKPHR | (Kme2)(Kpr)PHR |
| | (SEQ ID | |
| | NO. 6) | |
| H3K27me1K36me3 | KSAPATG | (pr)-(Kme1pr)SAPATGGV |
| | GVKKPHR | (Kme3)(Kpr)PHR |
| | (SEQ ID | |
| | NO. 6) | |
| H3K27me2K36me0 | KSAPATG | (pr)-(Kme2)SAPATGGV |
| | GVKKPHR | (Kpr)(Kpr)PHR |
| | (SEQ ID | |
| | NO. 6) | |
| H3K27me2K36me1 | KSAPATG | (pr)-(Kme2)SAPATGGV |
| | GVKKPHR | (Kme1pr)(Kpr)PHR |
| | (SEQ ID | |
| | NO. 6) | |
| H3K27me2K36me2 | KSAPATG | (pr)-(Kme2)SAPATGGV |
| | GVKKPHR | (Kme2)(Kpr)PHR |
| | (SEQ ID | |
| | NO. 6) | |
| H3K27me3K36me0 | KSAPATG | (pr)-(Kme3)SAPATGGV |
| | GVKKPHR | (Kpr)(Kpr)PHR |
| | (SEQ ID | |
| | NO. 6) | |
| H3K27me3K36me1 | KSAPATG | (pr)-(Kme3)SAPATGGV |
| | GVKKPHR | (Kme1pr)(Kpr)PHR |
| | (SEQ ID | |
| | NO. 6) | |
| H3K27ac1K36me0 | KSAPATG | (pr)-(Kac)SAPATGGV |
| | GVKKPHR | (Kpr)(Kpr)PHR |
| | (SEQ ID | |
| | NO. 6) | |
| H3K27ac1K36me1 | KSAPATG | (pr)-(Kac)SAPATGGV |
| | GVKKPHR | (Kme1pr)(Kpr)PHR |
| | (SEQ ID | |
| | NO. 6) | |
| H3K27ac1K36me2 | KSAPATG | (pr)-(Kac)SAPATGGV |
| | GVKKPHR | (Kme2)(Kpr)PHR |
| | (SEQ ID | |
| | NO. 6) | |
| H3K27ac1K36me3 | KSAPATG | (pr)-(Kac)SAPATGGV |
| | GVKKPHR | (Kme3)(Kpr)PHR |
| | (SEQ ID | |
| | NO. 6) | |
| H3.3K27me0K36me0 | KSAPATG | (pr)-(Kpr)SAPSTGGV |
| | GVKKPHR | (Kpr)(Kpr)PHR |
| | (SEQ ID | |
| | NO. 6) | |
| H3Y41ph0 | YRPGTVALR | (pr)-YRPGTVALR |
| | (SEQ ID | |
| | NO. 7) | |
| H3K56me0 | YQKSTELLIR | (pr)-YQ(Kpr)STELLIR |
| | (SEQ ID | |
| | NO. 8) | |
| H3K56me1 | YQKSTELLIR | (pr)-YQ(Kme1pr) |
| | (SEQ ID | STELLIR |
| | NO. 8) | |
| H3K79me0 | EIAQDFKTDLR | (pr)-EIAQDF(Kpr)TDLR |
| | (SEQ ID | |
| | NO. 9) | |
| H3K79me1 | EIAQDFKTDLR | (pr)-EIAQDF(Kme1pr) |
| | (SEQ ID | TDLR |
| | NO. 9) | |
| H3K79me2 | EIAQDFKTDLR | (pr)-EIAQDF(Kme2) |
| | (SEQ ID | TDLR |
| | NO. 9) | |
| Abbreviations: | | |
| Chemical | | |
| derivatives: | | |
| pr | Proprionyl | |
| ac | Acetyl | |
| me1 | Monomethyl | |
| me2 | Dimethyl | |
| me3 | Trimethyl | |
| ubq | ubiquityl | |
| | stub (GG) | |
| me0 = ac0 | Unmodified | |

An example of this data is presented in the heat map in FIG. 1. For Example, a specific MCS was assigned to Cluster A for high methylation of H3K27me levels, low levels of H3K27me1 and low levels of H3K36 me0/me1. In contrast, in cells such as SKM1, H3K27me3 levels are low, and these are assigned to a different MCS cluster (Cluster F).

### Example 2

### Histone Methylation Profiling of EZH2 Gain-of-Function Mutation

Cancer genome sequencing studies have identified recurrent mutations (or sequence variants) of epigenetic proteins but many of these alterations lack functional analysis by MCS (Ryan et al., Science 2012; 336:1513-1514). The method described above in Example 1 can functionally analyze sequence variants. This is useful as it provides a more accurate analysis than genotyping known EZH2 mutations as it is functionally based, and also provides a better methodology in analyzing previously uncharacterized EZH2 mutations.

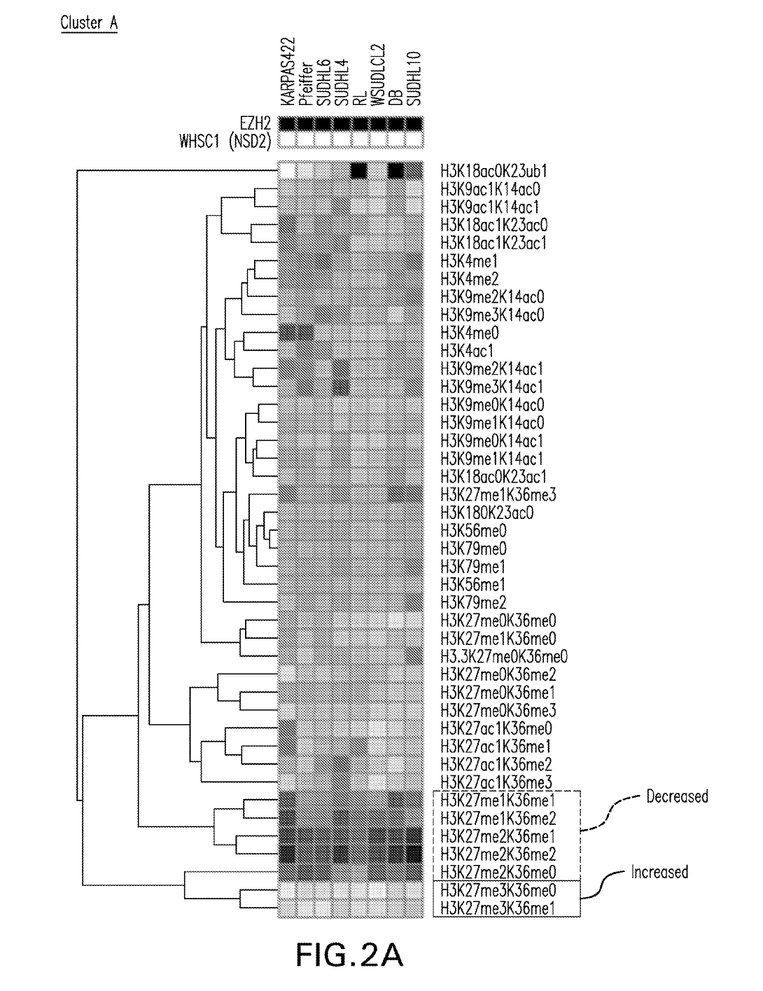

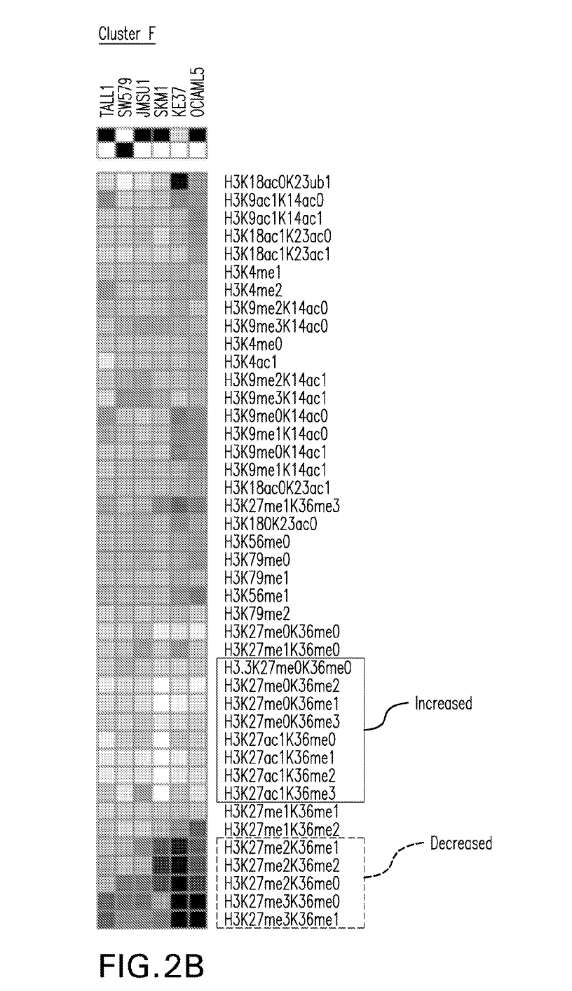

This is illustrated in FIG. 2A/B. Genotyping indicates that EZH2 variants; 1708M, G623D, H279Y and R685H are loss-of-function variants as they cluster closely with SKM1 cells which was previously shown to be EZH2 loss-of-function (Ernst et al., Nat. Genet. 2010 42:722-726). Cells with an EZH2 loss of function are predicted to be insensitive to EZH2 inhibitors, as the cancer cells have not become dependent on increased methyltransferase activity as seen in EZH2 gain of function mutations (e.g. EZH2 Y641N). An EZH2 loss of function mutation has a MCS methylation profile that is much different from that of an EZH2 gain of function profile. The MCS for loss of function shows very low levels of di and tri-methylation (me2/3) at lysine 27 in histone H3 (H3K27me 2/3). In contrast, the EZH2 gain of function mutations show very high levels of tri-methylation (H3K27me3) and very low levels of H3K27me1/2). Without being bound to any single theory, the EZH2 gain of function mutations are heterozygous, with the non-mutant copy of the gene “priming” histone methylation by adding a single methyl group to H3K27 (H3K27me1), with the gain of function mutation showing increased activity and increased methylation to produce high levels of H3K27me3.

This is shown in FIG. 2A, where the increase of in levels of H3K27me3 and the commensurate decrease in H3K27me2 and me1 are indicated. This MCS methylation profile is found in cells with EZH2 gain of function mutations. In FIG. 2B the opposite MCS is seen. The SKM1 cells genotype to a gain of function EZH2 mutation. However, by MCS, which is a functional assay, the level of H3K27me3 is decreased and the level of H3K27me0 is increased. Thus, SKM1 cells genotype to a gain of function mutation, but the MCS shows that they are functionally an EZH2 loss of function mutation, and therefore are resistant to EZH2 inhibitor treatment.

### Example 3

### Clustering EZH2 Methylation Differences

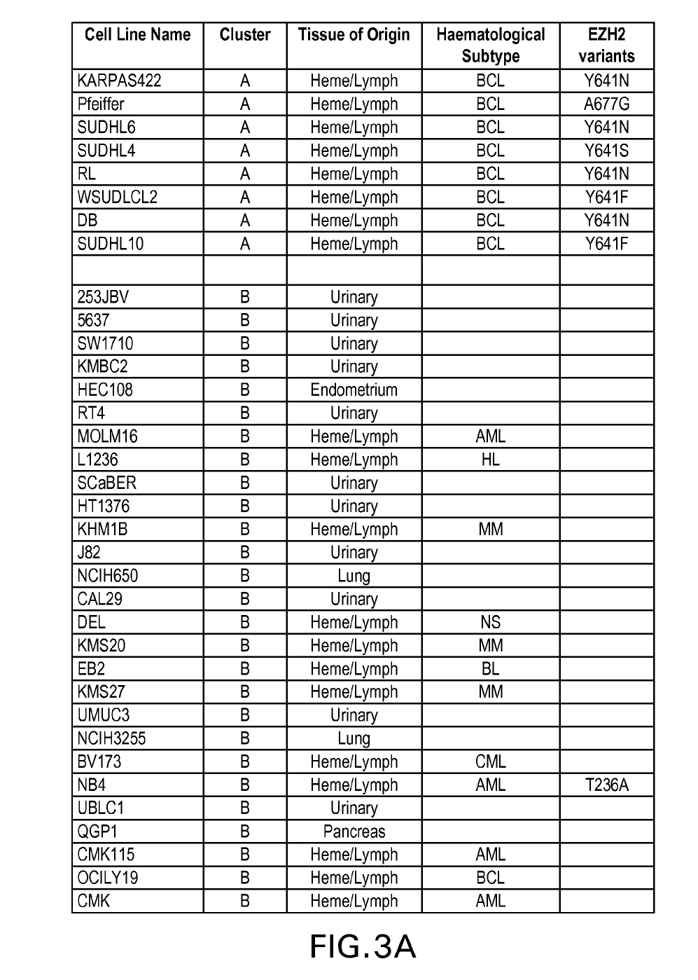

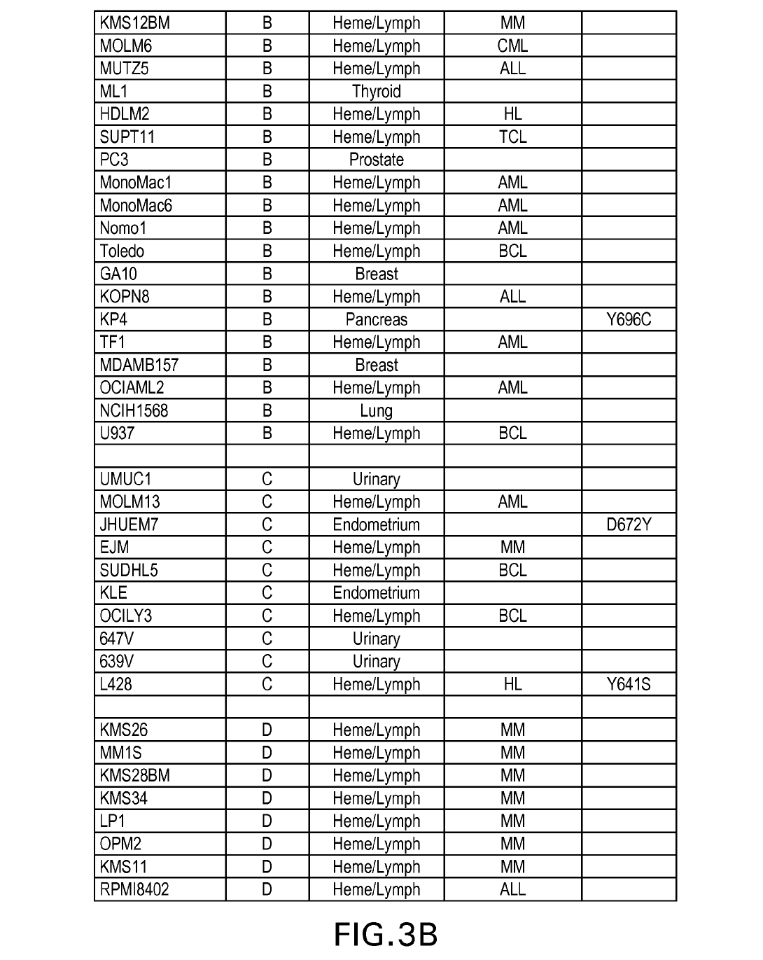

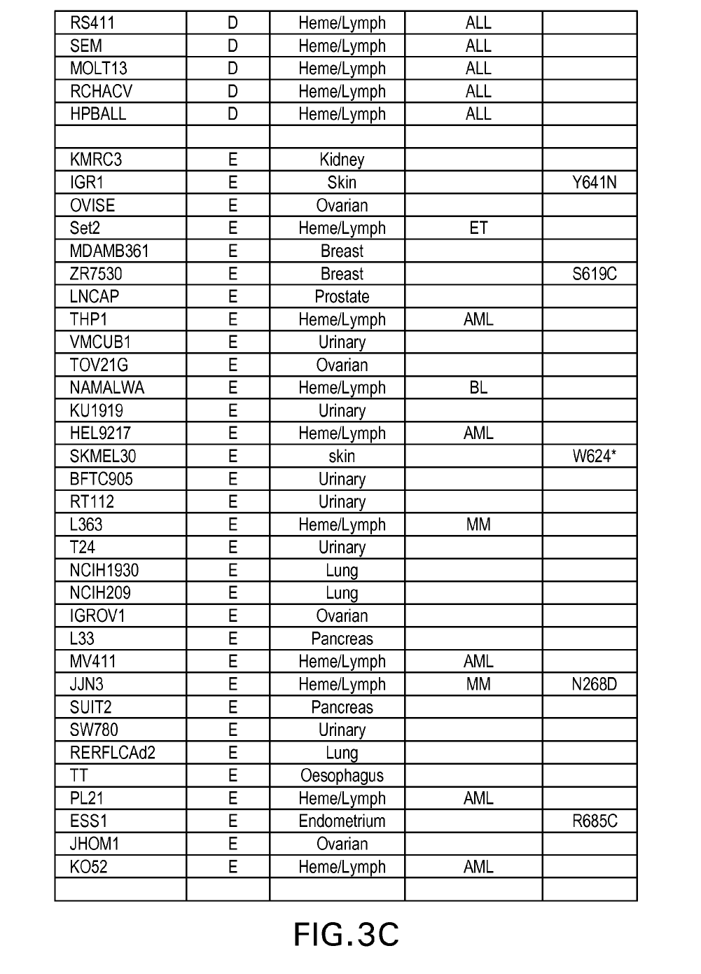

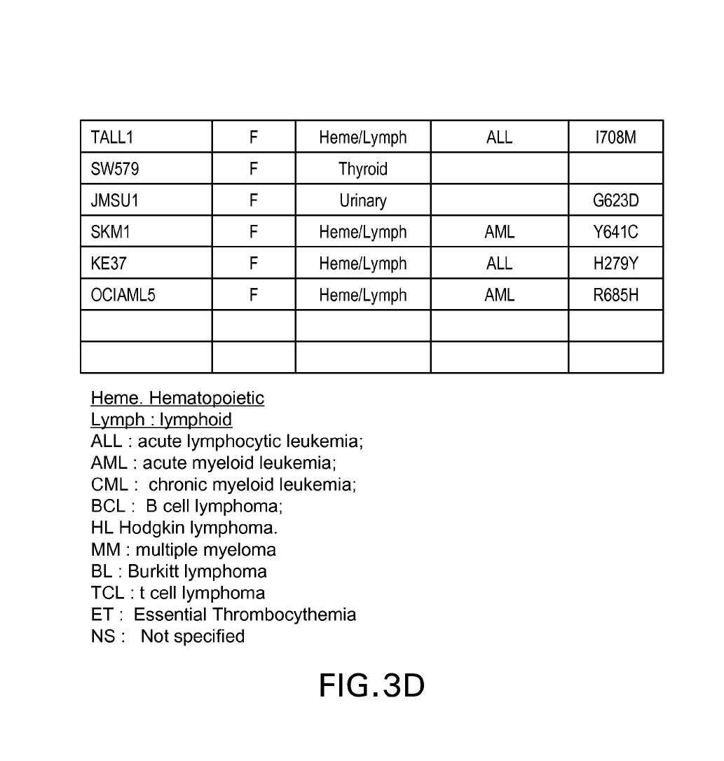

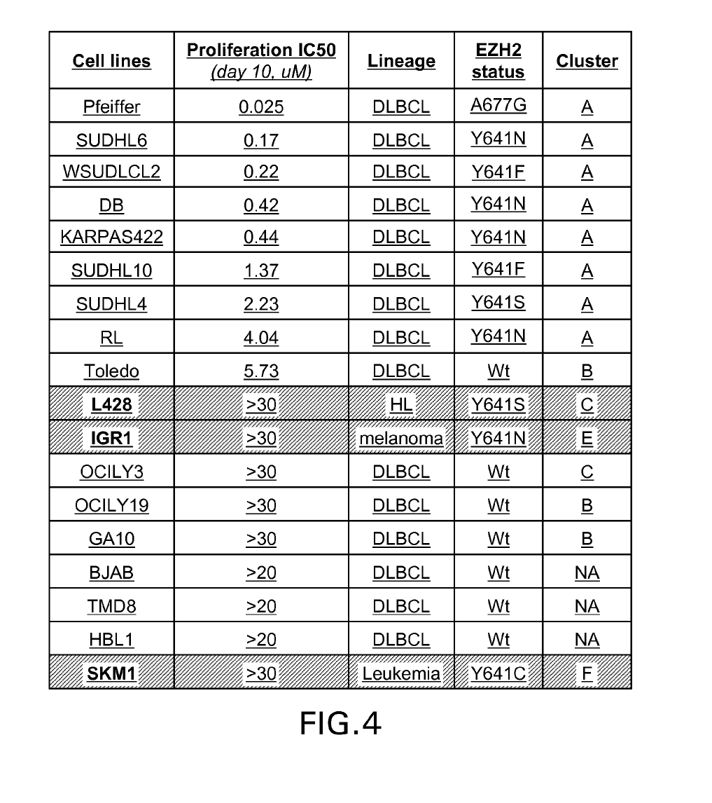

This Example shows that the method of MCS profiling can be used for patient stratification and provides a surprisingly more accurate result than genotyping. FIG. 3 is a table showing the cell line, the MCS cluster it was placed into and EZH2 mutation (if any) associated with that cell line. MCS cluster A is predicted to be sensitive to an EZH2 inhibitor, while MCS cluster F is predicted to be resistant. Testing this prediction is shown in FIG. 4, cancer cell lines with EZH2 gain-of-function mutations are predicted to be sensitive to EZH2 inhibitor treatment, and this is shown by the cell lines that have micromolar to sub-micromolar IC50. This is consistent with the genotyping of these cell lines as they all contain alterations at tyrosine 641 or alanine 677. These mutations are gain of function mutations which results in increased methyltransferase activity.

In contrast, 3 cell lines (IGR-1, L428 and SKM-1) contain EZH2 gain of function mutations by genotype (e.g. Y641 S), but cluster away from cluster A by MCS (see FIG. 1). By genotype these three lines contain EZH2 gain of function mutations, they are predicted to be sensitive to EZH2 inhibitors, but instead they are insensitive, with greater than 30 micromolar IC50.

The results in FIG. 4 show MCS is a more reliable biomarker to predict efficacy toward EZH2 inhibitor therapeutics. Again, without being bound to any one theory, these cells can be insensitive as they are heterozygous gain of function mutations and as such no “priming” of H3K27me1 occurs, so the EZH2 gain of function mutations have no substrate to act upon and no increased H3K27me3 is seen. Alternatively, while the genotyping predicts having an EZH2 enzyme with increased activity, the protein expression of this methyltransferase can be low, also resulting in lower levels of H3K27me3. Thus, the MCS profiling at H3K27me3 is a better readout of patient sensitivity, especially in predicting which patients will be insensitive to EZH2 inhibitors.

### Example 4

### Sensitive and Insensitive Cell Lines

B cell lymphoma cells were cultured using standard cell culture conditions. For example, lymphoma cells were cultured in RPMI-1640 (Invitrogen, cat #11875, Grand Island, N.Y.) supplemented with 15% FBS (Invitrogen, cat #10099-141, Grand Island, N.Y.) in humidified incubator at 37° C., 5% CO2. To assess the effect of EZH2 inhibitor on cell proliferation, exponentially growing cells were seeded at a density of 1×10⁵ cells/ml in 12-well plate (Corning, cat #CLS3513 Tewksbury, Mass.). Compounds were added after cell seeding at the indicated final concentration, and viable cell number was determined every 3-4 days continuously during the course of experiment using Vi-CELL (Beckman Coulter, Brea, Calif.). On days of cell counting, fresh growth media and compound were replenished and cells split back to a density of 1×10⁵ cells/ml. Total cell number and population doubling was then calculated.

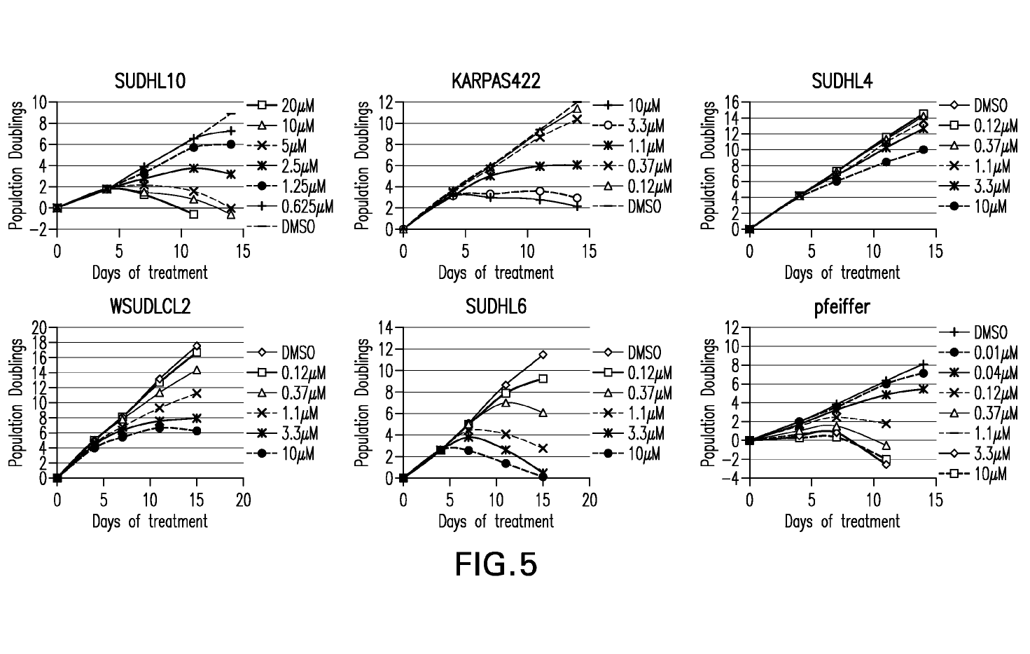

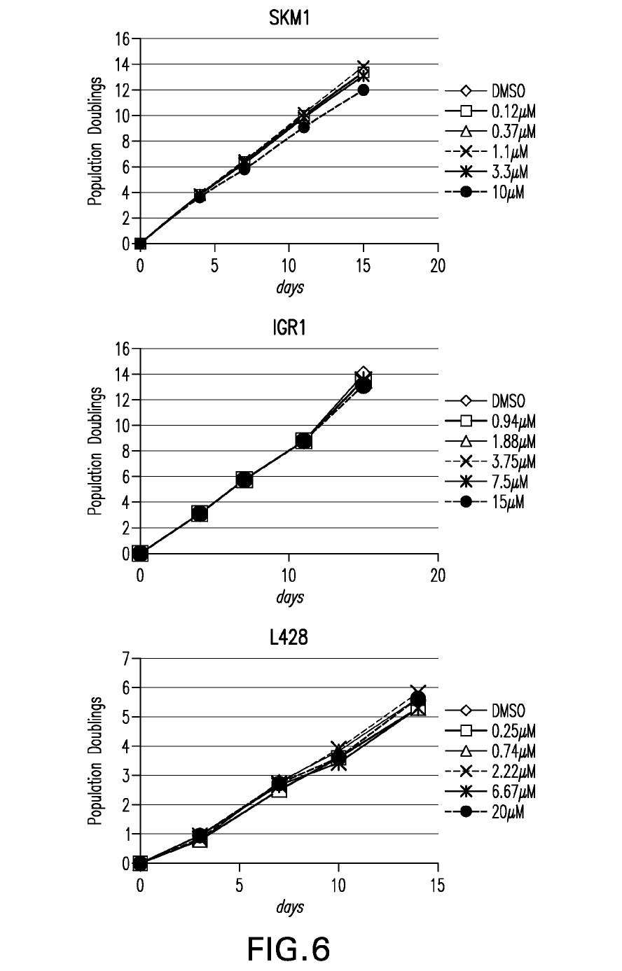

The differentiation between EZH2 inhibitor sensitive and insensitive is show in FIG. 5, where cells predicted to be sensitive by EZH2 genotyping and by MCS are shown to be sensitive when treated with an EZH2 inhibitor. FIG. 6 shows cells predicted to be sensitive by genotyping, but insensitive to EZH2 inhibitors by MCS with low levels of H3K27me3 are insensitive. The EZH2 inhibitor used was EI1 as disclosed in the structure above. Thus, MCS profiling is a valuable predictor of insensitivity to EZH2 inhibitors.

1. A method of detecting sensitivity of a cancer cell to an EZH2 inhibitor, the method comprising;
a) obtaining a cancer sample from a patient;
b) isolating histones from the cancer sample;
c) analyzing the histones by mass spectrometry to obtain a molecular chromatin signal (MCS); and
d) comparing the MCS of the cancer sample to a wild-type MCS in a non-cancerous or normal patient sample.

2. The method of claim 1, wherein the cancer cell contains a mutation in EZH2.

3. The method of claim 2, wherein the mutation in EZH2 is a change from alanine (A) at amino acid position 677 to a glycine (G); the mutation in EZH2 is a change from tyrosine (Y) at amino acid position 641 to the group consisting of serine (S), cysteine (C), phenylalanine (F), histidine (H) and asparagine (N) or the mutation in EZH2 is a change from alanine (A) at amino acid position 687 to a valine (V).

4. The method of claim 1, wherein the MCS is tri-methylation at histone H3, lysine 27 (H3K27me3).

5. The method of claim 4, wherein the H3K27me3 level is low or decreased, indicating insensitivity of the cancer cell to an EZH2 inhibitor.

6. The method of claim 1, wherein the cancer cell is selected from the group consisting of:
lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

7. The method of claim 1 further comprising: e) contacting cells of the cancer sample with an EZH2 inhibitor of the pyridinonyl substituted indolines class of molecules to determine sensitivity.

8. The method of claim 7, wherein the EZH2 inhibitor is 6-cyano-N-((4,6-dimethyl-2-oxo-1,2-dihydropyridin-3-yl)methyl)-1-(pentan-3-yl)-1H-indole-4-carboxamide.

9. A method of screening for an EZH2 inhibitor candidate, the method comprising:
a) contacting a cell containing an EZH2 gain of function mutation with an EZH2 inhibitor candidate;
b) isolating histones from the contacted cell;
c) analyzing the histones by mass spectrometry to obtain a molecular chromatin signal (MCS); and
d) comparing the reduction in H3K27me3 level in the EZH2 mutant cell contacted with the EZH2 inhibitor candidate with the H3K27me3 level in a normal or control cell and/or untreated cells containing the EZH2 mutation.

10. The method of claim 9, wherein the mutation in EZH2 is a change from alanine (A) at amino acid position 677 to a glycine (G); the mutation in EZH2 is a change from tyrosine (Y) at amino acid position 641 to the group consisting of serine (S), cysteine (C), phenylalanine (F), histidine (H) and asparagine (N) or the mutation in EZH2 is a change from alanine (A) at amino acid position 687 to a valine (V).

11. The method of claim 10, wherein the cell containing an EZH2 mutation is selected from the group consisting of: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

12. The method of claim 9, further comprising: e) contacting the EZH2 mutant cell with a molecule from the pyridinonyl substituted indolines class of molecules and comparing sensitivity to the EZH2 inhibitor candidate.

13. The method of claim 12, wherein the molecule from the pyridinonyl substituted indolines class of molecules is 6-cyano-N-((4,6-dimethyl-2-oxo-1,2-dihydropyridin-3-yl)methyl)-1-(pentan-3-yl)-1H-indole-4-carboxamide.

14. Composition comprising H3K27me3 for use in diagnosis of cancer in a selected cancer patient population, wherein the cancer patient population is selected on the basis of containing an EZH2 mutation in a cancer cell sample obtained from said patients compared to a normal control cell sample.

15. The composition wherein the cancer sample is selected from the group consisting of is selected from the group consisting of: lymphomas, prostate cancer, breast carcinoma, plasma cell myeloma, leukemias, bladder carcinoma, colon cancer, cutaneous melanoma, ovarian cancers, renal cell carcinoma, gliomas, neuroblastomas, hepatocellular carcinoma, endometrial cancer, lung cancer, pancreatic cancer, gastric cancer, thyroid cancer, rhabdomyosarcoma, malignant rhabdoid cancers, synovial sarcoma and Ewing sarcoma.

16. A kit for predicting the insensitivity of a cancer patient for treatment with a EZH2 inhibitor comprising: i) means for detecting decreased levels of H3K27me3 by mass spectrometry; and ii) instructions how to use said kit.
